(19) United States
(12) Patent Application Publication
(10) Pub. No.: US 2025/0176905 A1
(43) Pub. Date: Jun. 5, 2025

(54) DEVICES AND METHODS FOR PREDICTING EFFECT OF A CARDIOVASCULAR MEDICATION ADMINISTRATION PROGRAM

(71) Applicant: LIVEMETRIC (MEDICAL) S.A., Luxembourg (LU)

(72) Inventors: Bat-Chen PELES YEHOSHUA, Pardes Hanna-Karkur (IL); Tomer BENTZION, Tel Aviv (IL); Ori HAY, Haifa (IL)

(21) Appl. No.: 18/844,971

(22) PCT Filed: Mar. 14, 2023
(86) PCT No.: PCT/IL2023/050263

§ 371 Date: Sep. 8, 2024

Related U.S. Application Data
(60) Provisional application No. 63/320,171, filed on Mar. 15, 2022.

(51) Int. Cl. A61B 5/00; A61B 5/021

(52) U.S. Cl. CPC A61B 5/4848; A61B 5/02116; A61B 5/02141; A61B 5/7275; A61B 5/742

(57) ABSTRACT

There is provided herein a method for evaluating the response to and/or the effectiveness of a cardiovascular medication administration program (CMAP), the method including providing a wearable device comprising a pressure sensor array, receiving at least one first signal associated with a first arterial pressure waveform of the patient obtained prior to the change in CMAP, receiving at least one second signal associated with a second arterial pressure waveform of the patient obtained after (or during) the change in CMAP, comparing the at least one first and second signals, thereby identifying differences between the first and second arterial pressure waveforms, and displaying the identified differences between the first and second arterial pressure waveforms.

### FIELD OF THE INVENTION

The present disclosure relates generally to prediction and evaluation of response to a cardiovascular medication administration program and/or the effectiveness thereof.

### BACKGROUND

Patients with cardiovascular conditions typically receive one or more medications of specific dosage and frequency of use, in what is known as the cardiovascular medication administration program. Often such patients with cardiovascular conditions may receive different or additional medications, or change in dosage thereof, in what is known as change of the cardiovascular medication administration program. Currently, methods for assessment of the effectiveness of cardiovascular medication administration program require special dedicated equipment only available in health care facilities. Existing non-invasive monitoring devices capable of measuring physiological characteristics (such as ECG, heart rate, blood pressure, or oxygen level) are not suitable for rapid assessment since they can identify effectiveness only after significant time period because they either provide spot measurements (e.g., blood pressure measured by cuffs) or incomplete information (e.g., heart rate).

Nevertheless, there is a need in the art for methods and/or devices for providing caregivers predictions regarding expected patients' response to cardiovascular medications by utilizing various blood pressure related parameters.

### SUMMARY

According to some embodiments, there are provided devices and methods for predicting effect and/or effectiveness of cardiovascular medication. According to some embodiments, the device and/or method may be configured to record blood pressure waveforms and analyze the changes in the shape of the waveform. According to some embodiments, the device and/or method may be configured to compute and/or predict a pressure waveform that would result after the patient is administered with a change to the cardiovascular medication administration program. According to some embodiments, the device and/or method may be configured to automatically diagnose and/or assess an effect of the medication (or change thereto) given to the patient. According to some embodiments, the device and/or method may be configured to provide visual comparison of the waveforms, pre- and/or post-a change, to the patient's cardiovascular medication administration program, has been made.

Advantageously, the methods and devices disclosed herein enable clinicians/physicians to have immediate information on the effects of a new medication and/or a change in the dosage or timing of their administration program (also referred to as the cardiovascular medication administration program) in patients with cardiovascular conditions.

According to some embodiments, the methods and devices disclosed herein enable clinicians/physicians to have immediate information on the effects of a new medication and/or a change in the dosage or timing of their administration program using a wearable device, such as, for example, a wristband, rather than using invasive methods.

Advantageously, the methods and devices disclosed herein enable the user (e.g., the clinicians and/or physicians) to check the effect of a medication, within a short period of time after the medication has been given to a patient. According to some embodiments, the methods and devices disclosed herein output a visually clear difference which can be seen even 30 seconds after the administration of the medication to the patient.

Advantageously, a quick assessment of medication effectiveness may increase the efficacy of treatment in patients with uncontrolled cardiovascular conditions such as hypertension, arrhythmias, since the methods and/or devices may suggest (or recommend) whether a specific medication is ineffective or existence of an underlying condition (i.e., for example, if the patient is not actually being treated by the medication that they are taking).

Advantageously, the methods and/or devices may output information regarding a patient's response to cardiovascular medications, a change in medication, and/or a change in dose of a medication, which may improve treatment of the patient by revealing the underlying effect of the medication (or change thereto), thereby helping control and/or treat the patient's condition.

According to some embodiments there is provided a method for predicting the response to of a cardiovascular medication administration program (CMAP), the method including: receiving at least one first signal associated with a first arterial pressure waveform of the patient obtained prior to the change in CMAP, and predicting, using a model, an estimated arterial pressure waveform in response to the change in cardiovascular medication administration program, based, at least in part, on the first arterial pressure waveform of the patient and the type of change in CMAP.

According to some embodiments there is provided a method for evaluating the response to and/or the effectiveness of a cardiovascular medication administration program, the method including: receiving at least one first signal associated with a first arterial pressure waveform of the patient obtained prior to the change in cardiovascular medication administration program, predicting, using a model, an estimated arterial pressure waveform in response to the change in cardiovascular medication administration program, based, at least in part, on the first arterial pressure waveform of the patient and the type of change in cardiovascular medication administration program, receiving at least one second signal associated with a second arterial pressure waveform of the patient obtained after the initiation of change in cardiovascular medication administration program, comparing the predicted estimated arterial pressure waveform and the at least one second signal, thereby identifying differences therebetween, and displaying the identified differences.

According to some embodiments there is provided a method for predicting and evaluating the response to and/or the effectiveness of a cardiovascular medication administration program (CMAP), the method including: providing a wearable device including a pressure sensor array, receiving at least one first signal associated with a first arterial pressure waveform of the patient obtained prior to the change in CMAP, predicting, using a model, an estimated arterial pressure waveform in response to the change in cardiovascular medication administration program, based, at least in part, on the first arterial pressure waveform of the patient and the type of change in CMAP, receiving at least one second signal associated with a second arterial pressure waveform of the patient obtained after (or during) the change in CMAP, comparing the at least one first and second signals with the predicted estimated arterial pressure waveform, thereby identifying differences between the first arterial pressure waveform, the second arterial pressure waveform, and the predicted estimated arterial pressure waveform, and displaying the identified differences between the first arterial pressure waveform, the second arterial pressure waveform, and the predicted estimated arterial pressure waveform.

According to some embodiments there is provided a device for predicting the response of a cardiovascular medication administration program, the device including: a wearable body including a pressure sensor array and configured to be worn by a patient, and a processor in communication with a non-transitory computer-readable storage medium, the storage medium has stored thereon one or more program codes executable by the processor to: receive at least one first signal associated with a first arterial pressure waveform of the patient obtained prior to the change in cardiovascular medication administration program, and predict, using a model, an estimated arterial pressure waveform in response to the change in cardiovascular medication administration program, based, at least in part, on the first arterial pressure waveform of the patient and the type of change in cardiovascular medication administration program.

According to some embodiments there is provided a device for evaluating the response to and/or the effectiveness of a cardiovascular medication administration program, the device including: a wearable body including a pressure sensor array and configured to be worn by a patient, and a processor in communication with a non-transitory computer-readable storage medium, the storage medium has stored thereon one or more program codes executable by the processor to: receive at least one first signal associated with a first arterial pressure waveform of the patient obtained prior to the change in cardiovascular medication administration program, predict, using a model, an estimated arterial pressure waveform in response to the change in cardiovascular medication administration program, based, at least in part, on the first arterial pressure waveform of the patient and the type of change in cardiovascular medication administration program, receive at least one second signal associated with a second arterial pressure waveform of the patient obtained after the initiation of change in cardiovascular medication administration program, and compare the predicted estimated arterial pressure waveform and the at least one second signal, thereby identifying differences therebetween.

According to some embodiments there is provided a device for predicting and evaluating the response to and/or the effectiveness of a cardiovascular medication administration program (CMAP), the device including: a wearable body including a pressure sensor array and configured to be worn by a patient, and a processor in communication with a non-transitory computer-readable storage medium, the storage medium has stored thereon one or more program codes executable by the processor to: receive at least one first signal associated with a first arterial pressure waveform of the patient obtained prior to the change in CMAP, predict, using a model, an estimated arterial pressure waveform in response to the change in CMAP, based, at least in part, on the first arterial pressure waveform of the patient and the type of change in CMAP, receive at least one second signal associated with a second arterial pressure waveform of the patient obtained after (or during) the change in cardiovascular medication administration program, and compare the at least one first and second signals with the predicted estimated arterial pressure waveform, thereby identifying differences between the first arterial pressure waveform, the second arterial pressure waveform, and the predicted estimated arterial pressure waveform.

According to some embodiments, the method further includes outputting a recommended cardiovascular medication administration program based, at least in part, on the estimated arterial pressure waveform.

According to some embodiments, the first signal is acquired over about 1 minute to 24 hours.

According to some embodiments, the method further includes comparing the predicted estimated arterial pressure waveform and the at least one first signal, thereby identifying differences therebetween and displaying the identified differences.

According to some embodiments, the method further includes outputting a suggestion for a change in CMAP based, at least in part, on the comparison of the estimated arterial pressure waveform and the at least one first signal.

According to some embodiments, the type of change in cardiovascular medication administration program includes any one or more of: beginning a new treatment, change in type of medication, change in dosage of a medication, and change in timing of a medication, changing an administration regime, or any combination thereof.

According to some embodiments, estimating the estimated arterial pressure waveform is based, at least in part, on metadata associated with demographic data and/or medical history of the patient and/or the at least one first signal.

According to some embodiments, estimating the estimated arterial pressure waveform is based, at least in part, on the intended medication and/or dosage of the cardiovascular medication administration program.

According to some embodiments, the model includes a cardiovascular physiological model.

According to some embodiments, the cardiovascular physiological model includes any one or more of a Windkessel model, 0D models, 1D models, 2D models, one or more simulations of blood system, hemodynamic models, averaged expected response calculations, mathematical models, or any combination thereof.

According to some embodiments, the model includes a machine learning model.

According to some embodiments, the machine learning model includes any one or more of a regression model, neural networks, (deep) convolutional networks, support vector regressor, or any combination thereof.

According to some embodiments, the method further includes displaying the first signal and/or the predicted arterial pressure waveform includes one or more visual representation techniques using visual representation techniques.

According to some embodiments, the visual representation techniques are selected from: a two dimensional attractor, graphs of waveform signals, graphs of the difference in the signals, attractor reconstruction, Hilbert-Huang spectrum analysis with empirical mode decomposition (EMD), and the difference between waveform characteristics.

According to some embodiments, the medication associated with the cardiovascular medication administration program includes any one or more of one or more artery dilators (Vasodilators), beta blockers, Alpha blockers, Calcium channel blockers, Diuretics, Angiotensin-converting enzyme (ACE) inhibitors, Angiotensin receptor blockers (ARBs), Central Agonists, or any combination thereof.

According to some embodiments, the method further includes outputting a suggestion for a change in CMAP based, at least in part, on the comparison of the estimated arterial pressure waveform and the at least one second signal.

According to some embodiments, the at least one first signal is continuous.

According to some embodiments, the at least one second signal is continuous.

According to some embodiments, the type of change in cardiovascular medication administration program includes any one or more of: beginning a new treatment, change in type of medication, change in dosage of a medication, and change in timing of a medication, changing an administration regime, or any combination thereof.

According to some embodiments, the method further includes preprocessing the at least one first signal and/or the at least one second signal and/or the predicted estimated arterial pressure waveform, wherein the preprocessing includes dividing at least a portion of the least one signal into a plurality of segments, wherein each segment includes a heart cycle.

According to some embodiments, the preprocessing includes calculating the blood pressure from the at least one first signal and/or the at least one second signal and/or the predicted estimated arterial pressure waveform.

According to some embodiments, the method further includes normalizing the at least one signal and/or the at least one second signal and/or the predicted estimated arterial pressure waveform based, at least in part, on the blood pressure.

According to some embodiments, the method further includes normalizing the at least one signal and/or the at least one second signal and/or the predicted estimated arterial pressure waveform based, at least in part, on a length of time between beats.

According to some embodiments, estimating the estimated arterial pressure waveform is based, at least in part, on metadata associated with demographic data and/or medical history of the patient.

According to some embodiments, wherein estimating the estimated arterial pressure waveform is based, at least in part, on the intended medication and/or dosage of the cardiovascular medication administration program.

According to some embodiments, the model includes a cardiovascular physiological model.

According to some embodiments, the cardiovascular physiological model includes any one or more of a Windkessel model, 0D models, 1D models, 2D models, one or more simulations of blood system, hemodynamic models, averaged expected response calculations, mathematical models, or any combination thereof.

According to some embodiments, the model includes a machine learning model.

According to some embodiments, the machine learning model includes any one or more of a regression model, neural networks, (deep) convolutional networks, support vector regressor, or any combination thereof.

According to some embodiments, the method further includes displaying the first signal and/or the predicted arterial pressure waveform includes one or more visual representation techniques using visual representation techniques.

According to some embodiments, the visual representation techniques are selected from: a two dimensional attractor, graphs of waveform signals, graphs of the difference in the signals, attractor reconstruction, Hilbert-Huang spectrum analysis with empirical mode decomposition (EMD), and the difference between waveform characteristics.

According to some embodiments, the time difference between initiation of the change in cardiovascular medication administration program and the time in which the at least one second signal is obtained is lower than 30 seconds.

According to some embodiments, the time difference between initiation of the change in cardiovascular medication administration program and the time in which the at least one second signal is obtained is arbitrary.

According to some embodiments, the time difference between initiation of the change in cardiovascular medication administration program and the time in which the at least one second signal is obtained varies and correlates to a type of medication associated with the cardiovascular medication administration program.

According to some embodiments, the medication associated with the cardiovascular medication administration program includes any one or more of one or more artery dilators (Vasodilators), beta blockers, Alpha blockers, Calcium channel blockers, Diuretics, Angiotensin-converting enzyme (ACE) inhibitors, Angiotensin receptor blockers (ARBs), Central Agonists, or any combination thereof.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1A and FIG. 1B show isometric and side view schematic illustrations of a device for evaluating the response to and/or the effectiveness of a cardiovascular medication administration program, in accordance with some embodiments of the present invention;

FIG. 1C shows a schematic illustration of a device for evaluating the response to and/or the effectiveness of a cardiovascular medication administration program, with a terminal for displaying assessment result for clinician, in accordance with some embodiments of the present invention;

FIG. 2A shows a schematic block diagram of a system for evaluating the response to and/or the effectiveness of a cardiovascular medication administration program, in accordance with some embodiments of the present invention;

FIG. 2B shows a schematic block diagram of an exemplary system for evaluating the response to and/or the effectiveness of a cardiovascular medication administration program, in accordance with some embodiments of the present invention;

FIG. 3 shows a flow chart of the steps of a method for evaluating the response to and/or the effectiveness of a cardiovascular medication administration program, in accordance with some embodiments of the present invention;

FIG. 4 shows an exemplary blood pressure waveform and pulse detection applied to an exemplary signal, in accordance with some embodiments of the present invention;

FIG. 5A and FIG. 5B show exemplary blood pressure waveform of pulses just before administration (red) and 20 seconds after administration of medication (blue) and just before administration (red) and 100 seconds after administration of medication (blue), in accordance with some embodiments of the present invention;

FIG. 6A and FIG. 6B show an exemplary single pulse segment before (A) and after (B) pulse height normalization, respectively, in accordance with some embodiments of the present invention;

FIG. 7 shows a graph of an exemplary demonstration of decomposition of single heartbeat pulse (black) into gaussian waveforms, in accordance with some embodiments of the present invention;

FIG. 8 shows an exemplary pulse interval variability of a zoom-in portion of the blood pressure waveform of pulses just before administration (red) and 100 seconds after administration of medication (blue), of FIG. 5B, in accordance with some embodiments of the present invention;

FIG. 9A and FIG. 9B show an exemplary prevalence of irregular heartbeats before and after administration, and the standard deviation thereof, respectively, in accordance with some embodiments of the present invention;

FIG. 10A and FIG. 10B show two exemplary two-dimensional (2D) attractors of the first signal (red) and the second signal (blue) of different patients, in accordance with some embodiments of the present invention;

FIG. 11 shows a flow chart of the steps of a method for predicting and evaluating the response to and/or the effectiveness of a cardiovascular medication administration program using a physiological model, in accordance with some embodiments of the present invention;

FIG. 12 shows a flow chart of the steps of a method for predicting and evaluating the response to and/or the effectiveness of a cardiovascular medication administration program using a physiological model, in accordance with some embodiments of the present invention; and

FIG. 13 shows a flow chart of the steps of a method for predicting and evaluating the response to and/or the effectiveness of a cardiovascular medication administration program using a physiological model, in accordance with some embodiments of the present invention.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments a device is provided for evaluating the effectiveness of and/or the response to a cardiovascular medication administration program (CMAP). According to some embodiments, the device may be a wearable device which may be capable of measuring pressure waveforms from the wrist of a subject wearing it. According to some embodiments, the device may include a wearable body including a pressure sensor array and configured to be worn by a subject. According to some embodiments, the device may include a processor in communication with a non-transitory computer-readable storage medium, the storage medium has stored thereon one or more program codes (or one or more algorithms).

According to some embodiments, the device may be configured to send a clinician the at least one first signal associated with a first arterial pressure waveform of the patient obtained prior to the change in cardiovascular medication administration program. According to some embodiments, the device may be configured to send a clinician at least one second signal associated with a second arterial pressure waveform of the patient obtained after (or during) the change in cardiovascular medication administration program. According to some embodiments, and as described in greater detail elsewhere herein, the device may send the at least one first signal associated with a first arterial pressure waveform and the at least one second signal associated with a second arterial pressure waveform of the patient to a display, thereby enabling the clinician to compare the at least one first and second signals.

According to some embodiments, the one or more algorithms may be configured to receive at least one first signal associated with a first arterial pressure waveform of the patient obtained prior to the change in cardiovascular medication administration program. According to some embodiments, the one or more algorithms may be configured to receive at least one second signal associated with a second arterial pressure waveform of the patient obtained after (or during) the change in cardiovascular medication administration program. According to some embodiments, the one or more algorithms may be configured to compare the at least one first and second signals, thereby identifying differences between the first and second arterial pressure waveforms. According to some embodiments, the one or more algorithms may be configured to display the identified differences between the first and second arterial pressure waveforms.

According to some embodiments there are provided devices and/or methods for evaluating the response to cardiovascular medications, which may be configured to address the problem of assessing the cardiovascular medication administration program effectiveness. According to some embodiments, the device may be configured to acquire a continuous non-invasive arterial (such as, e.g., radial) pressure signal (or in other words, a signal associated with the blood pressure, such as, e.g., in the form of a pressure waveform). According to some embodiments, the device and/or method disclosed herein may enable acquisition of the pressure waveforms (for example, in the format of a continuous pressure signal) prior to medication administration program or change thereof, as well as after (and/or during) medication was administered (or a change in administration dosage was made). Accordingly, by comparing the waveform signals and/or their characteristics prior to and post administration of medication, the devices and methods disclosed herein may provide data associated with whether the change in medication administration affected the patient. According to some embodiments, the devices and methods disclosed herein may provide data associated with how and/or how much the change in medication administration affected the patient, such as the patient's cardiovascular physiology.

According to some embodiments there are provided devices and/or methods for predicting the pressure waveform shape, which may be configured to address the problem of estimating the expected change following the beginning of cardiovascular medication administration program and/or changes within the cardiovascular medication administration program thereof. According to some embodiments, the method may output a prediction of the estimated arterial pressure waveform after a new and/or changed cardiovascular medication administration program.

According to some embodiments, predicting the estimated arterial pressure waveform after a new and/or changed cardiovascular medication administration program may include taking into account that different medications may have different effects on the body physiology, and may therefore have different impact on the cardiovascular system. According to some embodiments, predicting the estimated arterial pressure waveform after a new and/or changed cardiovascular medication administration program may include providing additional patient's medical information such as health status, comorbidities, past and current conditions, test results. According to some embodiments, the method may include predicting the estimated arterial pressure waveform while taking into account that medications related to the cardiovascular system such as artery dilators (Vasodilators), beta blockers, Alpha blockers, Calcium channel blockers, Diuretics, Angiotensin-converting enzyme (ACE) inhibitors, Angiotensin receptor blockers (ARBs), Central Agonists, that affects the blood flow, thus modifying the blood pressure waveform shape.

According to some embodiments, the method may include calculating the estimated arterial pressure waveform using a physiological model. According to some embodiments, the physiological model may include, but is not limited to, any one or more of a Windkessel model, one or more 0D, 1D, and/or 2D models, and/or one or more one-dimensional (1D models), or any combination thereof. Each possibility is a separate embodiment. According to some embodiments, the physiological model may include implementing one or more models of physiological mechanisms and/or analyses associated with how a specific medication is supposed to affect the arterial pressure waveform. According to some embodiments, the physiological model may be based, at least in part, on the first obtained signal associated with the arterial pressure waveform before the change in CMAP was made. According to some embodiments, the physiological model may be based, at least in part, on metadata associated with the patient's medical record, such as, demographics and/or medical history. According to some embodiments, the method may include using the metadata and/or the first signal to estimate one or more initial parameters of the physiological model. According to some embodiments, the method may include applying one or more algorithms configured to receive the at least one first signal and compute the medication effects on the shape of the waveforms and/or its characteristics according to the expected physiological effects, using the physiological model.

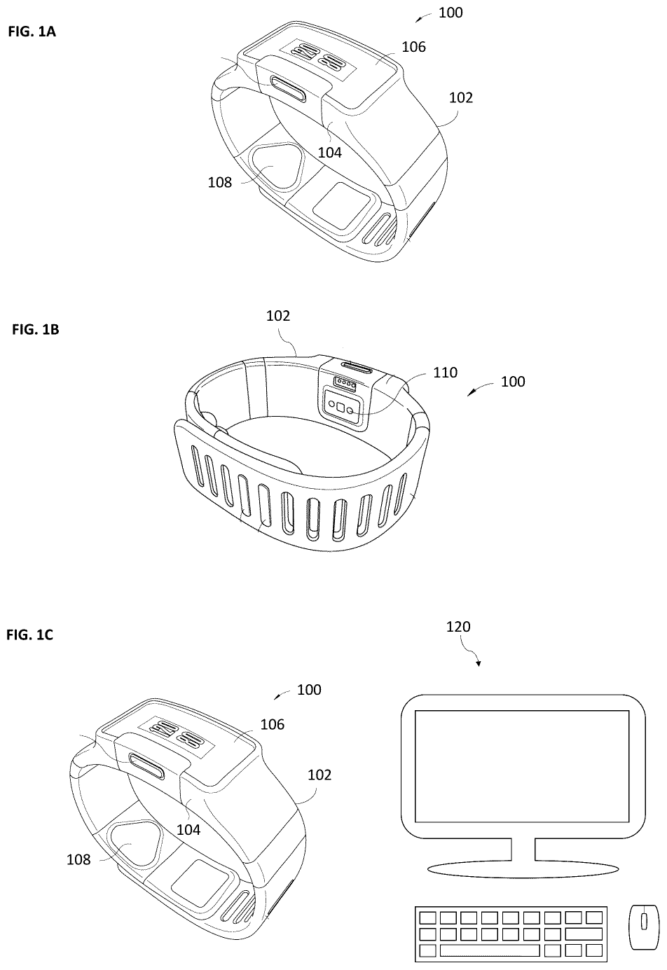

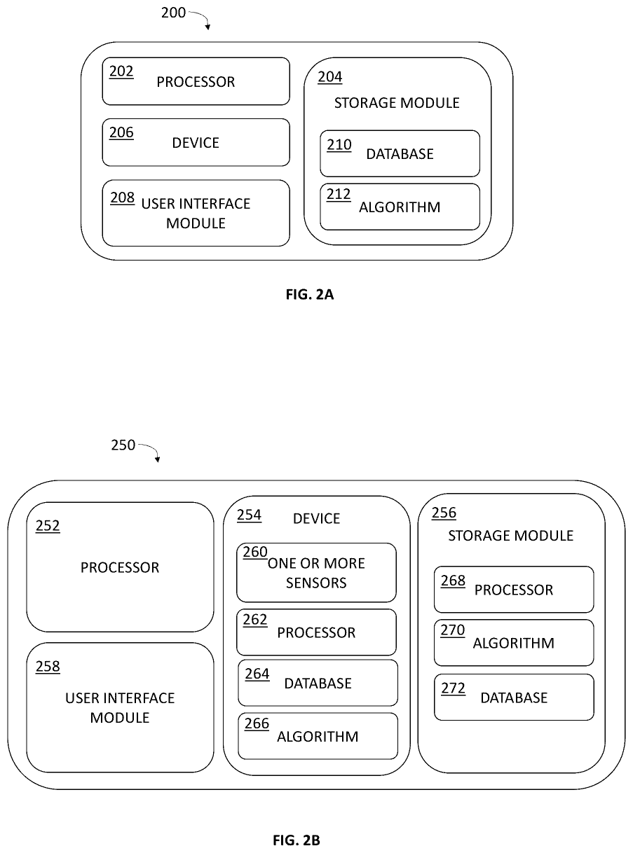

Reference is made to FIG. 1A and FIG. 1B, which show isometric and side view schematic illustrations of a device for evaluating the response to and/or the effectiveness of a cardiovascular medication administration program, in accordance with some embodiments of the present invention, and to FIG. 1C, which shows a schematic illustration of a device for evaluating the response to and/or the effectiveness of a cardiovascular medication administration program, with a terminal for displaying assessment result for clinician, in accordance with some embodiments of the present invention.

Reference is made to FIG. 2A, which shows a schematic block diagram of a system for evaluating the response to and/or the effectiveness of a cardiovascular medication administration program, in accordance with some embodiments of the present invention, and to FIG. 2B, which shows a schematic block diagram of an exemplary system for evaluating the response to and/or the effectiveness of a cardiovascular medication administration program, in accordance with some embodiments of the present invention.

According to some embodiments, the device 100 for evaluating the response to and/or the effectiveness of a cardiovascular medication administration program may include a wearable body 102. According to some embodiments, the wearable body 102 may include a display 106 (such as, for example, a viewable OLED screen, etc.), which may be mounted in a housing 104. According to some embodiments, the wearable body 102 and/or the housing 104 may include a processor (or in other words, a CPU) and a storage module in communication therewith. According to some embodiments, the communication between the processor and the storage module may be wired and/or wireless. According to some embodiments, the wearable body may include any one or more of one or more buttons, switches or dials, a band (and/or one or more straps), and/or a fastening mechanism configured to fasten the wearable body to the subject. According to some embodiments, the wearable body may include one or more pressure sensors configured to sense pressure of the radial and/or ulnar arteries.

According to some embodiments, the wearable body 102 may include a sensor array 108 configured to sense the pressure waveform from one or more blood vessels of the subject. According to some embodiments, the sensor array 108, which may include one or more pressure sensors, is positioned such that the one or more pressure sensors are positioned against the wrist of the subject. According to some embodiments, when the wearable body 102 is fastened to the subject, the sensor array 108 may be positioned on (or near) at least one of the radial, ulnar and brachial arteries. According to some embodiments, the wearable body 102 and/or the sensor array 108 may be configured to apply medium pressure to any one or more of the radial, ulnar and brachial arteries (i.e., for example, a pressure that is significantly less than the systolic pressure but enough to sense the pressure waveform). According to some embodiments, the wearable body 102 may include one or more additional sensors 110 such as, for example, an optical sensor or an ECG sensor.

According to some embodiments, the device 100 for evaluating the response to and/or the effectiveness of a cardiovascular medication administration program may include a wearable body 102 and accompanying computed device 120 that may include a processing system and user interface to display result.

According to some embodiments, the systems 200/250 for evaluating the response to and/or the effectiveness of a cardiovascular medication administration program may include the device 254/206/100. According to some embodiments, the systems 200/250 may include one or more processors 202/252/262/268 in communication with the device 254/206/100, wherein the one or more processors 202/252/262/268 may be configured to receive signals from the pressure sensor array 108 and/or additional sensors 110. According to some embodiments, the one or more sensors 260 may include one or more sensors of the pressure sensor array 108. According to some embodiments, the one or more sensors 260 may include one or more of the additional sensors 110. According to some embodiments, the one or more processors 202/252/262/268 may be in communication with a storage module 204/256 (or in other words, a non-transitory computer-readable storage medium). According to some embodiments, the storage module 204/256 may include a data module. According to some embodiments, the storage module 204/256 may include one or more algorithms 212/266/270 configured to receive one or more signals from the pressure sensor array 108 and/or additional sensors 110. According to some embodiments, the storage module 204 may include (or be in communication with) a database 210 configured to store user inputted data and/or recorded signals from the pressure sensor array 108 and/or additional sensors 110.

According to some embodiments, the storage module 256 may include (or be in communication with) a database 264/272 configured to store user inputted data and/or recorded signals from the one or more sensors 260. According to some embodiments, the processor 202 and/or one or more algorithms 212 may be configured to control the amount of data collected from the pressure sensor array 108 and/or additional sensors 110. According to some embodiments, the one or more processors 252/262/268 and/or one or more algorithms 266/270 may be configured to control the amount of data collected from the one or more sensors 260. According to some embodiments, the processor 202 and/or one or more algorithms 212 may be configured to control the frequency of data collection from the pressure sensor array 108 and/or additional sensors 110. According to some embodiments, the one or more processors 252/262/268 and/or one or more algorithms 266/270 may be configured to control the frequency of data collection from the one or more sensors 260. According to some embodiments, the one or more processors 202/252/262/268 and/or one or more algorithms 212/266/270 may be configured to schedule one or more of the start time(s) and/or the duration of the received signals.

According to some embodiments, the system 200/250 may include a user interface module 208/258 configured to receive input from a user and/or output data to the user. According to some embodiments, the user interface module 208/258 may include a display monitor (or screen) configured to display the comparison between first and/or second signal and/or predicted signal, as described in greater detail elsewhere herein. According to some embodiments, the user interface module 208/258 may include any one or more of a keyboard, a mouse, one or more buttons, a touchscreen, and the like. According to some embodiments, the system 200/250, processor 202 and/or user interface module 204 may be embedded (or integrated with) the device 100. According to some embodiments, the system 200/250, the one or more processors 262 and/or user interface module 258 may be embedded (or integrated with) the device 100/206/254. According to some embodiments, the system 200/250, the one or more processors 202/252/262/268 and/or user interface module 204/256 may be part of an accessory device, such as a mobile phone, a PC, a cloud server, and the like. According to some embodiments, the accessory device may be configured to do at least a portion of the processing. According to some embodiments, the accessory device may be configured to present the received signals and/or the comparison thereof to the clinician. According to some embodiments, the accessory device may be configured to run the one or more algorithms. According to some embodiments, the accessory device may be configured to save data to the database, such as database 210.

According to some embodiments, the one or more algorithms may be configured to implement one or more methods for evaluating the response to and/or the effectiveness of a cardiovascular medication administration program as described herein.

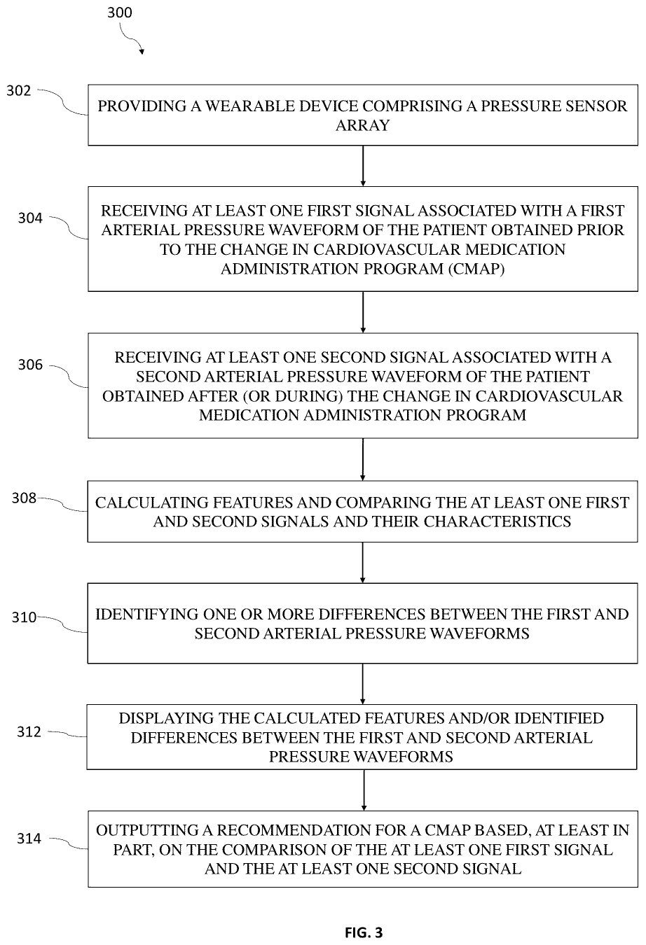

Reference is made to FIG. 3 shows a flow chart of the steps of a method for evaluating the response to and/or the effectiveness of a cardiovascular medication administration program, in accordance with some embodiments of the present invention.

According to some embodiments, at step 302, the method 300 for evaluating the response to and/or the effectiveness of a cardiovascular medication administration program may include providing a wearable device (such as, for example the device 100 of FIG. 1A and FIG. 1B) including a pressure sensor array. According to some embodiments, at step 304, the method 300 may include receiving at least one first signal associated with a first arterial pressure waveform of the patient obtained prior to the change in cardiovascular medication administration program (CMAP). According to some embodiments, at step 306, the method 300 may include receiving at least one second signal associated with a second arterial pressure waveform of the patient obtained after (or during) the change in cardiovascular medication administration program. According to some embodiments, at step 308, the method 300 may include calculating features and comparing the at least one first and second signals and their characteristics. According to some embodiments, at step 310, the method 300 may include identifying differences between the first and second arterial pressure waveforms based on their features and characteristics. According to some embodiments, at step 312, the method 300 may include displaying the calculated features and/or identified differences between the first and second arterial pressure waveforms. According to some embodiments, at step 314, the method 300 may include outputting a recommendation for a CMAP based, at least in part, on the comparison of the at least one first signal and the at least one second signal.

According to some embodiments, at step 304, the method 300 may include receiving at least one first signal associated with a first arterial pressure waveform of the patient obtained prior to the change in cardiovascular medication administration program (CMAP). According to some embodiments, receiving the at least one first signal may include recording the at least one first signal. According to some embodiments, at step 306, the method 300 may include receiving at least one second signal associated with a second arterial pressure waveform of the patient obtained after (or during) the change in cardiovascular medication administration program. According to some embodiments, receiving the at least one second signal may include recording the at least one second signal.

According to some embodiments, the medication associated with the cardiovascular medication administration program may include any one or more of one or more artery dilators (Vasodilators), beta blockers, Alpha blockers, Calcium channel blockers, Diuretics, Angiotensin-converting enzyme (ACE) inhibitors, Angiotensin receptor blockers (ARBs), Central Agonists, or any combination thereof.

According to some embodiments, recording may refer to a single blood pressure waveform acquisition of the at least one of the first and/or second signals. According to some embodiments, recording may refer to a blood pressure waveform acquisition over a short period of time (such as, for example, several seconds and/or several heartbeats). According to some embodiments, the recording may be blood pressure waveform acquisition for one or more seconds. According to some embodiments, recording may refer to a (continuous) blood pressure waveform acquisition over a long time (such as, for example, a range from one or more minutes to a plurality of days). According to some embodiments, recording may refer to multiple recordings, which may include one or more recordings over short periods of time, one or more recordings over long periods of time and/or any combination thereof.

According to some embodiments, the time difference between initiation of the change in cardiovascular medication administration program and the time in which the at least one second signal is obtained is lower than 30 seconds. According to some embodiments, the time difference between initiation of the change in cardiovascular medication administration program and the time in which the at least one second signal is obtained is lower than 60 seconds.

According to some embodiments, the time difference between initiation of the change in cardiovascular medication administration program and the time in which the at least one second signal is obtained is arbitrary. According to some embodiments, the time difference between initiation of the change in cardiovascular medication administration program and the time in which the at least one second signal is obtained is dependent on the type of medication. According to some embodiments, the time difference between initiation of the change in cardiovascular medication administration program and the time in which the at least one second signal is obtained is arbitrary within a short range, of about 30 seconds to several minutes. According to some embodiments, the time difference between initiation of the change in cardiovascular medication administration program and the time in which the at least one second signal is obtained is arbitrary within a long range, of about 1 to 5 days. According to some embodiments, the time difference between initiation of the change in cardiovascular medication administration program and the time in which the at least one second signal is obtained varies and correlates to a type of medication associated with the cardiovascular medication administration program.

According to some embodiments, the method 300 may include preprocessing the first and/or second signals. According to some embodiments, the preprocessing may include any one or more of best sensor selection, sensors fusion, noise detection, noise reduction, noise cancellation, signal enhancement, motion compensation, signal quality assessment, pulse detection, pulse normalization, heartbeat duration calculation, time normalization, anomalous pulse detection, anomalous pulse identification/classification, anomalous pulse filtering, blood pressure calculation or any combination thereof. According to some embodiments, noise reduction may include removing undesired noise from the first and/or second signals. According to some embodiments, noise reduction may include applying one or more filters to the first and/or second signals.

According to some embodiments, the method 300 may include preprocessing the plurality of segments associated with the first and/or second signals. According to some embodiments, the preprocessing may include a method for best sensor selection and/or sensor fusion. According to some embodiments, the best sensor selection and/or sensor fusion methods may include dividing the signal and/or segment. According to some embodiments, the method may include ranking (or grading) the divided signal and/or the segment, wherein the score with which the divided signal and/or the segment is ranked (or graded) is lowest for a non-physiological signal. According to some embodiments, the method may include ranking (or grading) the divided signal and/or the segment, wherein the score with which the divided signal and/or the segment is ranked (or graded) is highest for a physiological signal with high signal to noise ratio (SNR). According to some embodiments, the score may be indicative of which parts of the signal can be used for further evaluation. According to some embodiments, the score may be indicative of which sensor may be more reliable that the others. According to some embodiments, the sensors scores may be converted to weights used for fusion of multiple sensors into a single signal with high SNR.

According to some embodiments, the method 300 may include preprocessing the plurality of segments associated with the first and/or second signals. According to some embodiments, the preprocessing may include any one or more of noise detection, noise reduction, noise cancellation, signal enhancement, motion compensation, blood pressure calculation, signal quality assessment, pulse detection, pulse normalization, heartbeat duration calculation, time normalization, anomalous pulse detection, anomalous pulse identification, anomalous pulse filtering, anomalous pulse removal, or any combination thereof.

According to some embodiments, noise detection, noise reduction and/or noise removal may include applying one or more filters to the first and/or second signals. According to some embodiments, noise reduction, noise cancellation, adaptive noise removal, and/or signal enhancement may include removal of frequencies from the signals and/or the segments (which may make the evaluation more difficult), and/or any other method known to experts in the field. According to some embodiments, the signal enhancement may be configured to enhance the frequencies and/or portions of the signal and/or segment that may contain relevant data (e.g., for example, band and/or high pass filter).

According to some embodiments, motion compensation may include using an inertial measurement unit (IMU). According to some embodiments, the device may include an IMU in communication with the processor and may be configured to send one or more signals thereto. According to some embodiments, the method may include using one or more adaptive filters along with one or more IMU signals, and removing motion artifacts from the first and/or second signal and/or the plurality of segments.

According to some embodiments, preprocessing the first and/or second signals may include heartbeat segmentation (also referred to as “pulse detection”). According to some embodiments, heartbeat segmentation may include dividing at least a portion of the at least one first and/or second signals into a plurality of segments. According to some embodiments, each segment includes a heart cycle (and/or data associated with a heart cycle). According to some embodiments, the method may include dividing at least a portion of the pulses of the at least one first and/or second signals into a plurality of segments. According to some embodiments, comparing between the at least one first and second signals may include comparing between one or more values associated with the plurality of segments of the at least one first signal and one or more values associated with the plurality of segments of the at least one second signal. According to some embodiments, the plurality of segments includes at least three segments. According to some embodiments, the plurality of segments may include between about 3 and 150 segments, or any range therebetween. Each possibility is a separate embodiment. According to some embodiments, the plurality of segments may be equivalent sets (or subset) of pulses (or heartbeats). According to some embodiments, equivalent sets (or subsets) may include the same number of sets (or subset) of pulses (or heartbeats). According to some embodiments, equivalent sets (or subsets) may include the about same number of sets (or subset) of pulses (or heartbeats).

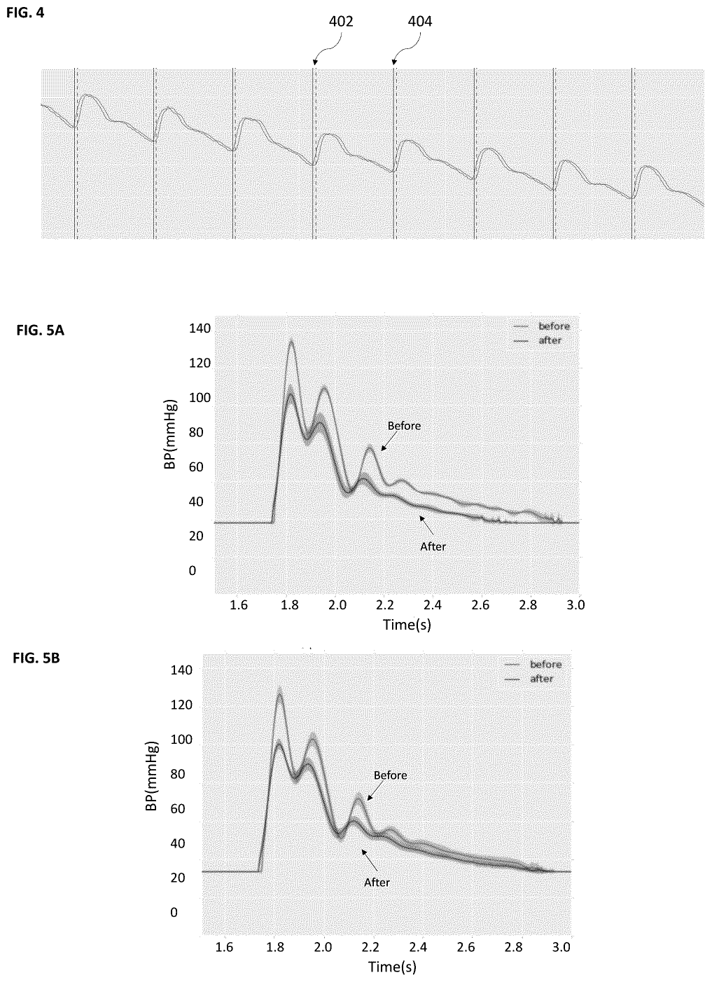

Reference is made to FIG. 4, which shows an exemplary blood pressure waveform and pulse detection applied to an exemplary signal, in accordance with some embodiments of the present invention. According to some embodiments, the pulse detection (or heartbeat segmentation) may include identifying a beginning (start) and/or end of a pulse within the first and/or second signal. According to some embodiments, the pulse detection may identify a pulse as the segment between the beginning of a first pulse (such as pulse starting at 402) and the beginning of the next consecutive pulse (such as pulse starting at 404), such as depicted in FIG. 4. According to some embodiments, the pulse detection (or heartbeat segmentation) may include identifying a beginning (start) and/or end of a plurality of pulses within the first and/or second signal.

According to some embodiments, blood pressure calculation may include calculating the blood pressure using data from the first and/or second signals. According to some embodiments, the blood pressure calculation may be used for normalization of the first and/or second signals.

According to some embodiments, the method may include analyzing at least one signal and/or segment using frequency domain and/or time (pulse) domain representation. According to some embodiments, the method may include identifying and/or calculating any one or more of a maximum frequency, an energy of specific frequency, a peak amplitude, a peak location (time of peak), a half width, at least one peak time interval, and at least one amplitude ratio, or any combination thereof, for any one or more of the first signal, second signal, and/or plurality of segments of the first and/or second signals. According to some embodiments, the method may include general pattern recognition algorithms, features based shape analysis (using peaks detection, gradients, correlation), machine learning or deep learning segmentation (using classification), for any one or more of the first signal, second signal, and/or plurality of segments of the first and/or second signals.

According to some embodiments, the preprocessing may include at least one heartbeat normalization method, normalizing the heartbeats within the first and/or second signals. According to some embodiments, the at least one heartbeat normalization method may include time normalization, normalizing the heartbeats within the first and/or second signals such that the length of a heartbeat is constant. According to some embodiments, the at least one heartbeat normalization method may include normalizing the at least one signal based, at least in part, on the calculated blood pressure. According to some embodiments, the at least one heartbeat normalization method may include normalizing the at least one signal based, at least in part, on the calculated heartbeat duration. According to some embodiments, normalizing the at least one signal includes normalizing the plurality of segments of the at least one signal.

According to some embodiments, normalizing the at least one first and/or second signal and/or the plurality of segments may include any one or more of a decomposition of triangular logarithmic, and/or gaussian waveforms. According to some embodiments, and as described in greater detail elsewhere herein, comparing the at least one first and second signals may include assessing at least one feature of the at least one normalized first and/or second signal.

Reference is made to FIG. 5A and FIG. 5B show exemplary blood pressure waveform of pulses just before administration (red) and 20 seconds after administration of medication (blue) and just before administration (red) and 100 seconds after administration of medication (blue), in accordance with some embodiments of the present invention.

According to some embodiments, and as described in greater detail elsewhere herein, the method may include outputting the blood pressure waveforms (or arterial line waveform) of the first signal and/or the second signal. According to some embodiments, and as described in greater detail elsewhere herein, the method may include comparing the first signal and the second signal and/or presenting the differences between the first signal and the second signal.

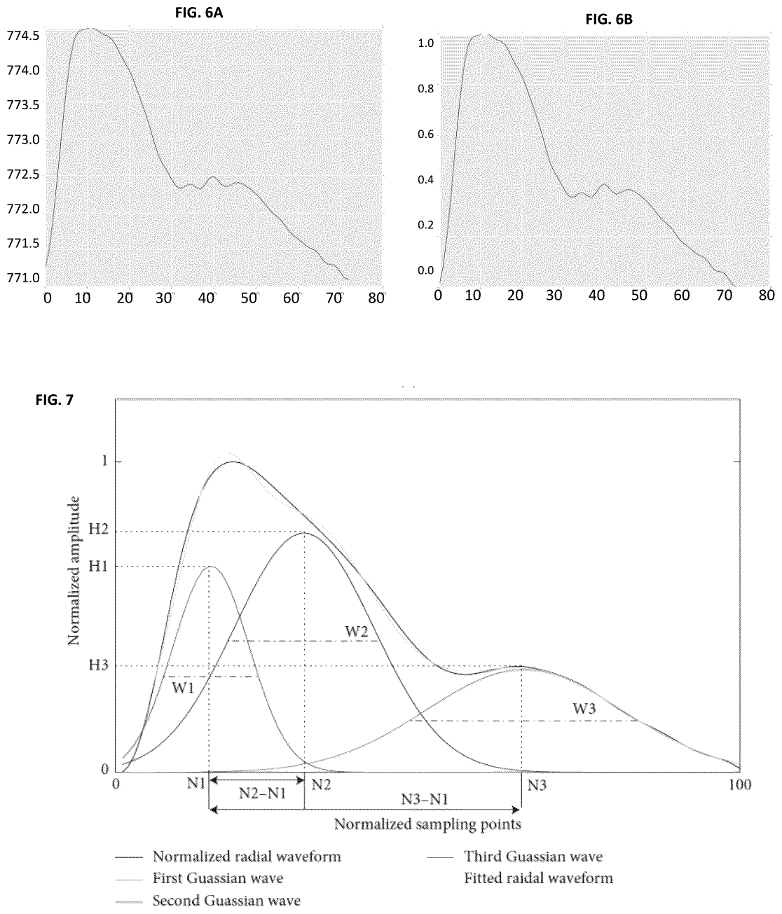

Reference is made to FIG. 6A and FIG. 6B, which show an exemplary single pulse segment before (A) and after (B) pulse height normalization, respectively, in accordance with some embodiments of the present invention. According to some embodiments, pulse normalization may include normalizing the pulse length. According to some embodiments, pulse normalization may include normalizing the pulse height (such as depicted in FIG. 6A and FIG. 6B).

According to some embodiments, the anomalous pulse detection, anomalous pulse identification, anomalous pulse filtering, and/or anomalous pulse removal may include comparing between signals and/or pulses within a signal. According to some embodiments, the method may include classifying the signals, segments, and/or individual pulses to normal physiological and/or anomalous and/or non-physiological signal using classification methods such as but not limited to machine learning, deep learning, correlation to a shape model, or simple thresholding. According to some embodiments, the anomalous pulse detection, anomalous pulse identification, anomalous pulse filtering, and/or anomalous pulse removal may include checking the signal and/or segment for one or more features which may be associated with non-physiological behavior. According to some embodiments, the method may include identifying one or more features which may be associated with non-physiological behavior within individual pulses. According to some embodiments, the method may include identifying and/or removing the signals, segments, and/or individual pulses which may include one or more features which may be associated with non-physiological behavior. According to some embodiments, non-physiological behavior may include any one or more of too many peaks, a pulse that is too long or too short (or in other words, above or below a predetermined threshold for the length of the pulse), a pulse that is relatively too long or too short (or in other words, above or below a threshold based on the previous pulses) a pulse amplitude that is too high or too low (or in other words, above or below a predetermined threshold for the amplitude of the pulse), a pulse amplitude that is relatively too high or too low (or in other words, above or below a threshold based on the previous pulses). According to some embodiments, the method may include calculating the threshold(s) by comparing two or more pulses of the first and/or second signals.

According to some embodiments, at step 308, the method 300 may include comparing the at least one first and second signals. According to some embodiments, comparing between the at least one first and second signals may include extracting, calculating, and/or assessing the at least one feature of the at least one first and/or second signals. According to some embodiments, the at least one feature may include any one or more features related to signal shape characteristics. According to some embodiments, the at least one feature may include any one or more of: at least one maximum value, at least one minimum value, a difference between at least one maximum value and at least one minimum value, an average between at least one maximum value and at least one minimum value, number of peaks, slopes between two or more extrema points, time of pulse's onset, time between two or more extrema points, ratio between times of two or more extrema points, energy, variability between pulses and/or any combination thereof.

Reference is made to FIG. 7, which shows a graph of an exemplary demonstration of decomposition of single heartbeat pulse (black) into gaussian waveforms, in accordance with some embodiments of the present invention. According to some embodiments, the method may include modeling the normalized pulse waveform using a decomposition of triangular, logarithmic, or gaussian waveforms, such as, for example, the decomposition of gaussian waveform as depicted in FIG. 7). According to some embodiments, the at least one feature of the normalized pulse waveform may include a peak amplitude, a peak location (time of peak), a half width, at least one peak time interval, and at least one amplitude ratio, variability between pulses or any combination thereof. According to some embodiments, the at least one frequency domain feature of any one or more of the first and/or second signals may include at least one of: a maximum frequency, an energy of specific frequency, a peak amplitude, a peak time position, a half width, at least one peak time interval, and at least one amplitude ratio, or any combination thereof. According to some embodiments, the at least one feature may include pulse interval variability. According to some embodiments, the at least one time domain feature may include the ratio between the heights of the valleys (or negative peaks) of the signals. According to some embodiments, the at least one feature may include the time of peaks in the signals.

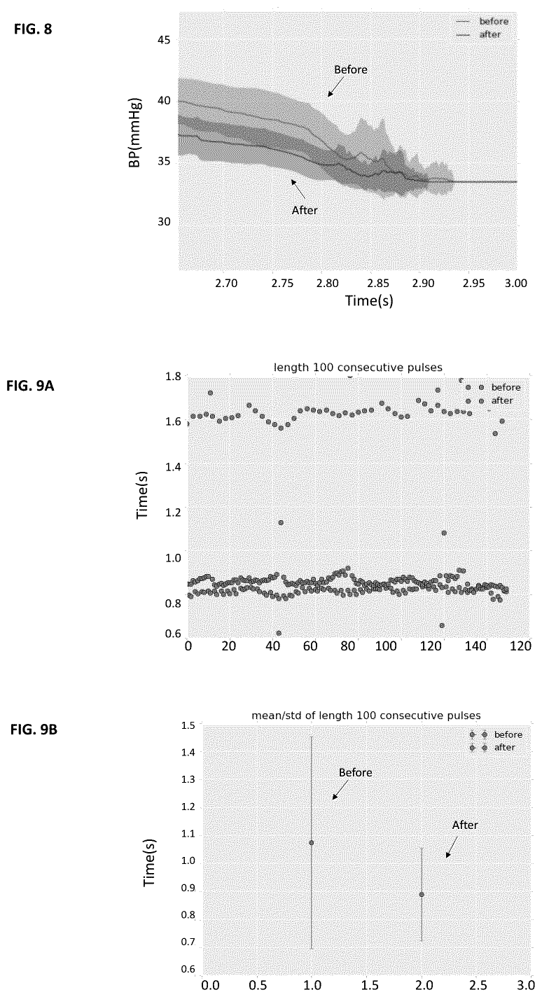

Reference is made to FIG. 8, which shows an exemplary pulse interval variability of a zoom-in portion of the blood pressure waveform of pulses just before administration (red) and 100 seconds after administration of medication (blue), of FIG. 5B, in accordance with some embodiments of the present invention.

According to some embodiments, such as depicted in FIG. 8, the variability of the pulse interval may decrease with use of some medications. According to some embodiments, the variability of the pulse interval may be measured at the end of the pulse, such as, for example, calculating the standard deviation using the equation:

$$\begin{matrix}std ( Δ t ) = \sqrt{\frac{1}{M} {\sum }_{m = 1}^{M} ( Δ {t}_{m} - \overline{Δ t} )} \\ std ( Δ {t}_{B} ) > s t d ( Δ {t}_{A} )\end{matrix}$$

wherein Δt is the pulse time interval, std is standard deviation, M is the number of pulses, B is the signal before administration (or the first signal) and A is the signal after administration (or the second signal).

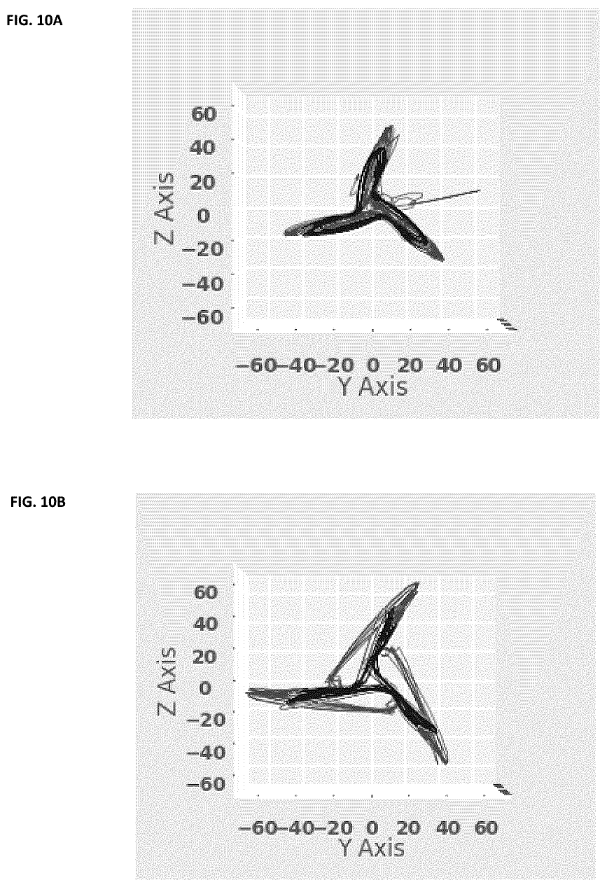

Reference is made to FIG. 9A and FIG. 9B, which show an exemplary prevalence of irregular heartbeats before and after administration, and the standard deviation thereof, respectively, in accordance with some embodiments of the present invention. According to some embodiments, the at least one feature may include the ratio (or proportion) of irregular beats. According to some embodiments, and as depicted by FIG. 9A and FIG. 9B, there may be a decrease in the proportion of irregular beats when some cardiovascular medication is administered.

According to some embodiments, the method may include the assessment of the at least one feature after the first and/or second signals and/or segments are normalized and/or preprocessed. According to some embodiments, comparing the at least one first and second signals (and/or the plurality of segments) may include assessing at least one feature of the at least one normalized first and/or second signal (and/or the plurality of segments).

According to some embodiments, comparing the at least one first and second signals (and/or the plurality of segments) may include transforming the at least one first and/or second signals (and/or the plurality of segments) to a first and/or second frequency domain, respectively, and then identifying and/or assessing at least one feature of the first and/or second frequency domains. According to some embodiments, comparing the at least one first and second signals includes comparing the change in one or more statistical attributes between pulses, such as average and/or variability, of at least one or more features of the at least one first and/or at least one second signals over time. According to some embodiments, comparing the at least one first and at least one second signals includes comparing change between each pulse waveform and/or one or more features thereof before and after the change in cardiovascular medication administration program.

According to some embodiments, at step 310, the method may include identifying one or more differences between the first and second arterial pressure waveforms. According to some embodiments, the method may include applying the first and/or second signals and/or the plurality of segments to an algorithm configured to identify the one or more features. According to some embodiments, the method may include applying the first and/or second signals and/or the plurality of segments to an algorithm configured to identify the one or more differences in the features. According to some embodiments, the method may include applying the features of the at least one first and/or at least one second signals to an algorithm configured to identify the one or more features. According to some embodiments, and as described in greater detail elsewhere herein, the algorithm may be a machine learning algorithm.

According to some embodiments, comparing the at least one first and second signals (and/or the plurality of segments) may include identifying one or more differences and/or classifying the one or more differences using one or more algorithms. According to some embodiments, the algorithm may be configured to compare between the at least one first and second signals (and/or the plurality of segments). According to some embodiments, the algorithm may include one or more classification algorithms such as machine learning, deep learning, expert system. According to some embodiments, the algorithm may be configured to classify the differences as being associated with an effective result of the CMAP. According to some embodiments, the algorithm may be configured to classify the differences as being associated with a positive effective result of the CMAP, a negative effective result of the CMAP, a placebo effect result of the CMAP, and no effective result of the CMAP. According to some embodiments, the algorithm may be configured to score (or rank) the effectiveness of change between the at least one first and second signals (and/or the plurality of segments), for example effectiveness score may be: negative (subject status is worse), ineffective (no change in subject status), somewhat effective (small beneficial change in subject status), effective (beneficial change in subject status), very effective (significant beneficial change in subject status). According to some embodiments, the algorithm may be configured to assess the one or more differences between the at least one first and second signals (and/or the plurality of segments).

According to some embodiments, when the CMAP is composed of more than one treatment procedure, the algorithm and method may be configured to identify and/or detect differences in the signals which may be associated with one of the treatment procedures (or in other words, identify an effect of a specific treatment procedure). According to some embodiments, the CMAP may be composed of two or more medications, such as, for example, taking one or more medications at two or more times a day. According to some embodiments, the algorithm and method may be configured to classify the differences as being associated with an effective result of the CMAP. According to some embodiments, at step 312, the method 300 may include displaying the calculated features and/or identified differences between the first and second arterial pressure waveforms.

According to some embodiments, the method includes displaying using one or more visual representation techniques. According to some embodiments, the one or more visual representation techniques may be selected from: a two-dimensional attractor, graphs of waveform signals, graphs of the difference in the signals, attractor reconstruction, Hilbert-Huang spectrum analysis with empirical mode decomposition (EMD), and the difference between waveform characteristics, or any combination thereof. For example, according to some embodiments, the visual representation techniques may include displaying the normalized pulse waveform before and after taking the medication.

According to some embodiments, the method may include displaying one or more waveform signals (or the first and/or second signals) together (such as, for example, on the same graph). According to some embodiments, the method may include displaying one or more waveform signals (or the first and/or second signals) separately (such as, for example, on separate, individual graphs) and displaying the difference between the signals (e.g., subtracted signal).

According to some embodiments, displaying the identified differences between the first and second arterial pressure waveforms may include using any one or more of pulse analysis methods, such as, for example, attractor reconstruction, or Hilbert-Huang spectrum analysis with empirical mode decomposition (EMD), and the difference between waveform characteristics, or any combination thereof. According to some embodiments, displaying the identified differences between the first and second arterial pressure waveforms may include displaying the difference between waveform characteristics (such as, for example, using graphs or bars).

According to some embodiments, displaying the identified differences between the first and second arterial pressure waveforms may include displaying changes to one or more individual features, such as, for example, a change in variability between pulses over time of the first and/or second signals (or the plurality of segments thereof).

According to some embodiments, the displaying may include a two-dimensional (2D) representation for identifying changes in the shape (or in other words, an attractor reconstruction). According to some embodiments, attractor reconstruction may include taking 3 points that are equally spaced apart, from each pulse. According to some embodiments, in attractor reconstruction, each of the 3 points is entered as coordinates to a three-dimensional (3D) plot. According to some embodiments, the 3D attractor may be viewed as a 2D attractor by viewing the 3D attractor from one corner, thereby creating a 2D attractor.

Reference is made to FIG. 10A and FIG. 10B, which show two exemplary two-dimensional (2D) attractors of the first signal (red) and the second signal (blue) of different patients, in accordance with some embodiments of the present invention. According to some embodiments, such as depicted in FIG. 10A and FIG. 10B, the data associated with the first signal and/or segments thereof is depicted in a different color (red) than the data associated with the second signal and/or segments thereof (which is depicted in blue).

According to some embodiments, at step 314, the method 300 may include outputting a recommendation for a CMAP based, at least in part, on the comparison of the at least one first signal and the at least one second signal. According to some embodiments, the method may include implementing an algorithm configured to output a recommendation for a CMAP. According to some embodiments, the algorithm may be configured to receive any one or more of the first signal, the second signal, the plurality of segments, one or more of the identified features, and/or one or more identified changes between the identified features of the first and/or second signal, or any combination thereof, as input (to the algorithm). According to some embodiments, the algorithm may be a machine learning algorithm. According to some embodiments, the algorithm may be configured to output a recommendation which may include any one of: maintaining at least a portion of the CMAP and changing at least a portion of the CMAP. According to some embodiments, the algorithm may be configured to output a recommendation which may include instructions to change at least a portion of the CMAP. According to some embodiments, the algorithm may be configured to output a recommendation including details of a specific change in the CMAP regime.

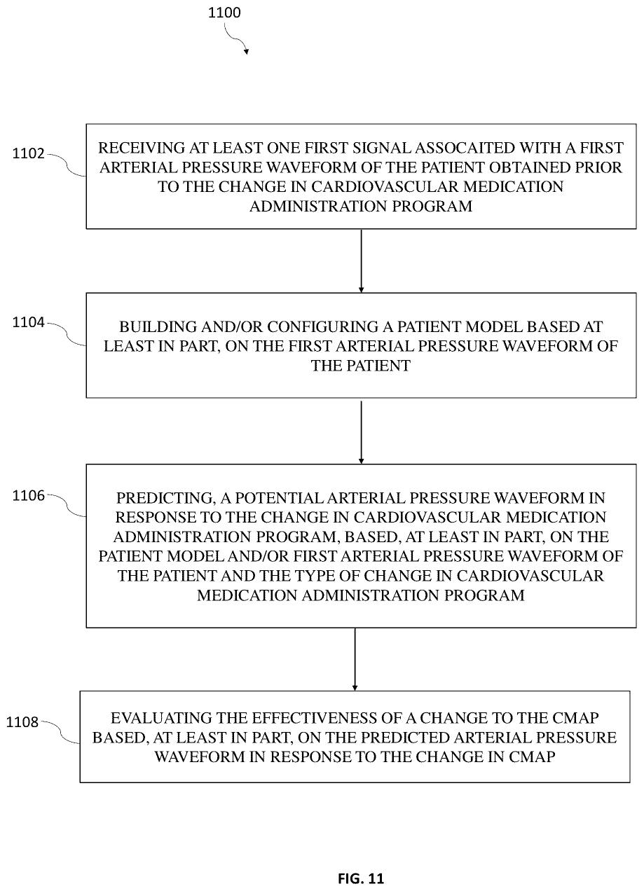

Reference is made to FIG. 11, which shows a flow chart of the steps of a method for predicting and evaluating the response to and/or the effectiveness of a CMAP using a physiological model, in accordance with some embodiments of the present invention. According to some embodiments, the method 300 may include one or more steps of the method 1100. According to some embodiments, the methods 300/1100 may include steps for predicting an estimated arterial pressure waveform in response to the change in cardiovascular medication administration program.

According to some embodiments, at step 1102 the method 1100 for evaluating the response to and/or the effectiveness of a CMAP of a patient may include receiving at least one first signal associated with a first arterial pressure waveform of the patient obtained prior to the change in cardiovascular medication administration program. According to some embodiments, at step 1104 the method 1100 for evaluating the response to and/or the effectiveness of a CMAP of a patient may include building and/or configuring a patient specific model (also referred to herein as “patient model”) based at least in part, on the first arterial pressure waveform of the patient. According to some embodiments, at step 1106, the method 1100 may include predicting, an estimated arterial pressure waveform in response to the change in cardiovascular medication administration program, based, at least in part, on the patient model and/or the first arterial pressure waveform of the patient and the type of change in cardiovascular medication administration program. According to some embodiments, at step 1108, the method 1100 may include evaluating the effectiveness of a change to the CMAP based, at least in part, on the predicted arterial pressure waveform in response to the change in CMAP.

According to some embodiments, the medication associated with the cardiovascular medication administration program includes any one or more of one or more artery dilators (Vasodilators), beta blockers, Alpha blockers, Calcium channel blockers, Diuretics, Angiotensin-converting enzyme (ACE) inhibitors, Angiotensin receptor blockers (ARBs), Central Agonists, or any combination thereof.

Advantageously, a method configured to predict an estimated arterial pressure waveform may enable physicians and/or users to make decisions regarding the current and/or changing cardiovascular medication administration program, based on the predicted estimated arterial pressure waveform, without having to administer the medication to the patient. In other words, the method may be used to prevent patients from taking medication which would not be beneficial to them, or even prevent patients from taking medication which would not be the best option, or most beneficial to them, relative to other optional medications.

According to some embodiments, the method 1100 may include providing a wearable device (such as, for example the device 100 of FIG. 1A, FIG. 1B and/or FIG. 1C) including a pressure sensor array. According to some embodiments, at step 1102, the method may include receiving at least one first signal associated with a first arterial pressure waveform of the patient obtained prior to the change in cardiovascular medication administration program from a wearable device, such as, device 100. According to some embodiments, the one or more processors 202/252/262/268 of device 100/206/254 (and/or in communication with the device 100/206/254) may be configured to execute method 1100. Each possibility is a separate embodiment. According to some embodiments, the processor of accompanying device 120 may be configured to execute method 1100.

According to some embodiments, at step 1102 the method 1100 may include receiving at least one first signal associated with a first arterial pressure waveform of the patient obtained prior to the change in cardiovascular medication administration program. According to some embodiments, the method may include acquiring the first signal over a period of time between about 1 minute to about one day (24 hours). According to some embodiments, the method may include recording the at least one first signal. According to some embodiments, recording may refer to a blood pressure waveform acquisition over a short period of time (such as, for example, several seconds and/or several heartbeats). According to some embodiments, the recording may be a blood pressure waveform acquisition for one or more seconds. According to some embodiments, recording may refer to a (continuous) blood pressure waveform acquisition over long time (such as, for example, a range from one or more minutes to a plurality of days). According to some embodiments, recording may refer to multiple recordings, which may include one or more recordings over short periods of time, one or more recordings over long periods of time and/or any combination thereof.

According to some embodiments, the method 1100 may include preprocessing the first signal. According to some embodiments, the preprocessing may include any one or more of noise detection, noise reduction, noise cancellation, signal enhancement, motion compensation, blood pressure calculation, signal quality assessment, pulse detection, pulse normalization, heartbeat duration calculation, time normalization, anomalous pulse detection, anomalous pulse identification, anomalous pulse filtering, anomalous pulse removal, beat segmentation, or any combination thereof, such as described in greater detail elsewhere herein. According to some embodiments, method may include analyzing at least one signal and/or segment using frequency domain and/or pulse domain, such as described in greater detail elsewhere herein.

According to some embodiments, the method 1100 may include inputting the first signal to one or more algorithms configured to predict the estimated arterial pressure waveform. According to some embodiments, the method 1100 may include inputting the first signal, the preprocessed signal, and/or segments associated with the first signal, to the one or more algorithms. According to some embodiments, the input to the one or more algorithms may be in the form of raw signals, preprocessed signals, and/or as one or more features thereof.

According to some embodiments, the method may include predicting, using a model, an estimated arterial pressure waveform in response to the change in cardiovascular medication administration program. According to some embodiments, the output of the estimated arterial pressure waveform may be in the form of raw signals, preprocessed-like signals, and/or as one or more features thereof. According to some embodiments, the model may be configured to generate the estimated arterial pressure waveform in response to the change in cardiovascular medication administration program based, at least in part, on the first arterial pressure waveform of the patient (or the first signal) and the type of change in cardiovascular medication administration program.

According to some embodiments, building and/or configuring the patient model may include one or more algorithms configured to receive the inputted first signal. According to some embodiments, the model may include a cardiovascular physiological model. According to some embodiments, the model may include any one or more of a Windkessel model, 0D models, 1D models, 2D models, one or more simulations of blood system, hemodynamic models, averaged expected response calculations, mathematical models, or any combination thereof. According to some embodiments, the model may be devoid of machine learning (or artificial intelligent) algorithms.

According to some embodiments, building and/or configuring the patient model may include a machine learning algorithm (or in other words, a machine learning model). According to some embodiments, the machine learning algorithm may include any one or more of one or more regression models, neural networks, (decp) convolutional networks, support vector regressor, or any combination thereof. According to some embodiments, the machine learning model may be trained to estimate the effects of medication (of a CMAP) after medication administration. According to some embodiments, the machine learning model may be trained to estimate the effects of medication (of a CMAP) and output the estimated arterial waveform.

According to some embodiments, the building and/or configuring of the patient model may include receiving metadata associated with demographic data and/or medical history of the patient. According to some embodiments, the metadata associated with demographic data and/or medical history of the patient may be inputted by the user (i.e., the patient and/or the physician). According to some embodiments, the metadata associated with demographic data and/or medical history of the patient may be retrieved automatically from a healthcare database (EHR-Electronic health record). According to some embodiments, the model may be configured to receive and/or store metadata associated with demographic data and/or medical history of the patient. According to some embodiments, the metadata may include any one or more of the age, gender, chronic medical conditions, current medical conditions, currently administered medications (and/or dosage thereof), and the like.

According to some embodiments, at step 1106 the method may include predicting, using the model of step 1104, and a prediction algorithm an estimated arterial pressure waveform in response to the change in cardiovascular medication administration program. According to some embodiments, the output of the estimated arterial pressure waveform may be in the form of raw signals, preprocessed-like signals, and/or as one or more features thereof. According to some embodiments, the prediction may be configured to generate the estimated arterial pressure waveform in response to the change in cardiovascular medication administration program based, at least in part, on the first arterial pressure waveform of the patient (or the first signal) and the type of change in cardiovascular medication administration program.

According to some embodiments, at step 1106 the prediction algorithm may include one or more of the following: adjusting the physiological model parameters based on the proposed CMAP, a machine learning prediction algorithm (or in other words, machine learning regression model). According to some embodiments, the machine learning algorithm may include any one or more of one or more regression models, neural networks, (deep) convolutional networks, support vector regressor, or any combination thereof. According to some embodiments, the machine learning model may be trained to estimate the effects of medication (of a CMAP) on the subject's physiological model after medication administration and adjusting the physiological model parameters. According to some embodiments, the machine learning model may be trained to estimate the effects of medication (of a CMAP) after medication administration. According to some embodiments, the machine learning model may be trained to estimate the effects of medication (of a CMAP) and output the estimated arterial waveform.

According to some embodiments, at step 1106 the prediction algorithm may be configured to predict the estimated arterial pressure waveform in response to the change in cardiovascular medication administration program (or in other words, the intended change in CMAP) using the metadata associated with demographic data and/or medical history of the patient.

According to some embodiments, the method may include receiving data associated with the intended medication and/or dosage thereof. According to some embodiments, the model may be configured to predict the estimated arterial pressure waveform in response to an intended change in cardiovascular medication administration program. According to some embodiments, the model may be configured to predict the estimated arterial pressure waveform in response to an intended change in CMAP based, at least in part, on the data associated with the intended medication and/or dosage thereof.

Advantageously, using the methods disclosed herein, the predicted estimated arterial pressure waveform can be presented, together with the signal even before the medication is given to the subject, thereby showing the patient and/or physician the estimated outcome of administrating a specific medication and/or dosage thereof.

According to some embodiments, the method may include displaying the predicted estimated arterial pressure waveform and the at least one first signal. According to some embodiments, the method may include comparing the predicted estimated arterial pressure waveform and the at least one first signal, thereby identifying differences therebetween. According to some embodiments, the method may include displaying the identified differences between the predicted estimated arterial pressure waveform and the at least one first signal.

According to some embodiments, the method includes displaying using one or more visual representation techniques. According to some embodiments, the one or more visual representation techniques may be selected from: a two-dimensional attractor, graphs of waveform signals, graphs of the difference in the signals, attractor reconstruction, Hilbert-Huang spectrum analysis with empirical mode decomposition (EMD), and the difference between waveform characteristics

According to some embodiments, the method may include displaying one or more waveform signals (or the first and/or predicted waveform) together (such as, for example, on the same graph). According to some embodiments, the method may include displaying one or more waveform signals (or the first and/or predicted waveform) separately (such as, for example, on separate, individual graphs) and display the difference in the signals (e.g., subtracted signal).

According to some embodiments, displaying the identified differences between the first and the predicted arterial pressure waveforms may include using any one or more of pulse analysis methods, such as, for example, attractor reconstruction, or Hilbert-Huang spectrum analysis with empirical mode decomposition (EMD), or any combination thereof. According to some embodiments, displaying the identified differences between the first and the predicted arterial pressure waveforms may include displaying the difference between waveform characteristics (such as, for example, using graphs or bars).

According to some embodiments, displaying the identified differences between the first and the predicted arterial pressure waveforms may include displaying changes to one or more individual features, such as, for example, a change in variability between pulses over time of the first and/or the predicted signals (or the plurality of segments thereof). According to some embodiments, the displaying may include a two-dimensional (2D) representation for identifying changes in the shape (or in other words, an attractor reconstruction). According to some embodiments, the 3D attractor may be viewed as a 2D attractor as described in greater detail elsewhere herein.

According to some embodiments, the model may be configured to output a recommended cardiovascular medication administration. According to some embodiments, the method may include outputting a recommended cardiovascular medication administration program (or in other words, a suggestion) based, at least in part, on the estimated arterial pressure waveform. According to some embodiments, the method may include outputting a suggestion for a change in CMAP based, at least in part, on the comparison of the estimated arterial pressure waveform and the at least one first signal. According to some embodiments, the type of change in cardiovascular medication administration program includes any one or more of: beginning a new treatment, change in type of medication, change in dosage of a medication, and change in timing of a medication, changing an administration regime, or any combination thereof.

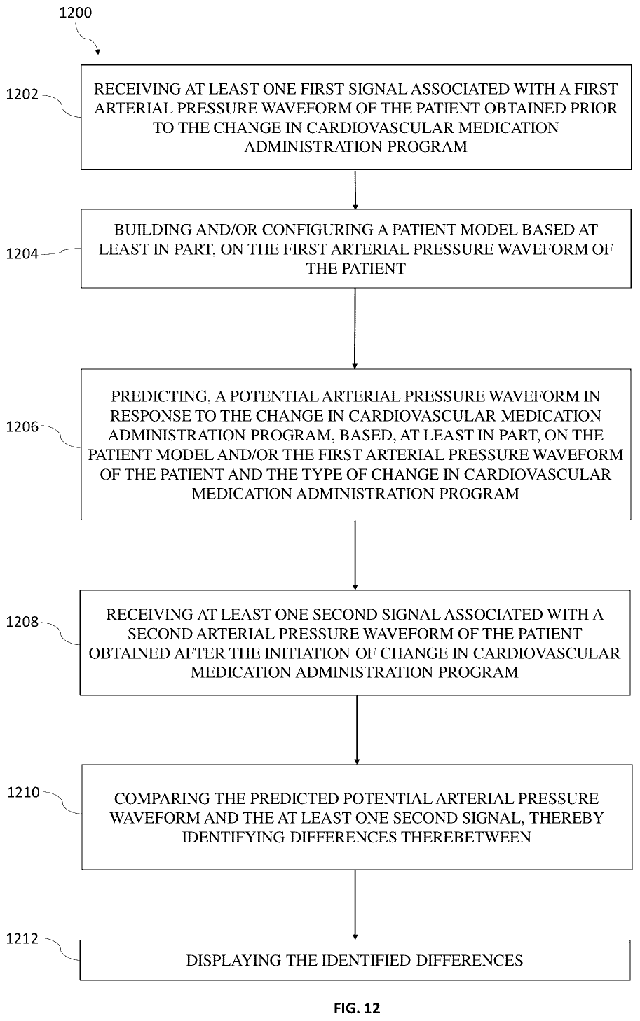

Reference is made to FIG. 12, which shows a flow chart of the steps of a method for predicting and evaluating the response to and/or the effectiveness of a cardiovascular medication administration program using a physiological model, in accordance with some embodiments of the present invention. According to some embodiments, the methods 300/1100 may include one or more steps of the method 1200. According to some embodiments, the method 1200 may include one or more steps of methods 300/1100.

According to some embodiments, at step 1202, the method 1200 may include receiving at least one first signal associated with a first arterial pressure waveform of the patient obtained prior to the change in cardiovascular medication administration program. According to some embodiments, at step 1204, the method may include building and/or configuring a patient model based at least in part, on the first arterial pressure waveform of the patient. According to some embodiments, at step 1206, the method may include predicting, an estimated arterial pressure waveform in response to the change in cardiovascular medication administration program, based, at least in part, on patient model and/or the first arterial pressure waveform of the patient and the type of change in cardiovascular medication administration program. According to some embodiments, at step 1208, the method may include receiving at least one second signal associated with a second arterial pressure waveform of the patient obtained after the initiation of change in cardiovascular medication administration program. According to some embodiments, at step 1210, the method may include comparing the predicted estimated arterial pressure waveform and the at least one second signal, thereby identifying differences therebetween. According to some embodiments, at step 1212, the method may include displaying the identified differences between the predicted estimated arterial pressure waveform and the at least one second signal.

Advantageously, a method configured to predict an estimated arterial pressure waveform (which estimates how the patient would respond to a cardiovascular medication administration program) and compare it to a second arterial pressure waveform of the patient obtained after the initiation of change in cardiovascular medication administration program, enables the patient and/or physician to identify whether the CMAP that was administered has effected the subject in a predictable manner, and thus enables the physician to administer other CMAP if needed.

According to some embodiments, the method 1200 may include providing a wearable device (such as, for example the device 100 of FIG. 1A and FIG. 1B) including a pressure sensor array. According to some embodiments, the method may include receiving at least one first signal and/or second signal from a wearable device, such as, device 100. According to some embodiments, the processor of device 100 may be configured to execute method 1200.

According to some embodiments, at step 1202, the method 1200 may include receiving at least one first signal associated with a first arterial pressure waveform of the patient obtained prior to the change in cardiovascular medication administration program. According to some embodiments, receiving the at least one first signal may include recording the at least one first signal. According to some embodiments, recording may refer to a single blood pressure waveform acquisition of the first signal. According to some embodiments, recording may refer to a blood pressure waveform acquisition over a short period of time (such as, for example, several seconds and/or several heartbeats). According to some embodiments, the recording may be blood pressure waveform acquisition for one or more seconds. According to some embodiments, recording may refer to a (continuous) blood pressure waveform acquisition over long time (such as, for example, a range from one or more minutes to a plurality of days). According to some embodiments, recording may refer to multiple recordings, which may include one or more recordings over short periods of time, one or more recordings over long periods of time and/or any combination thereof.

According to some embodiments, the method 1200 may include preprocessing the first signal. According to some embodiments, the preprocessing may include any one or more of noise detection, noise reduction, noise cancellation, signal enhancement, motion compensation, blood pressure calculation, signal quality assessment, pulse detection, pulse normalization, heartbeat duration calculation, time normalization, anomalous pulse detection, anomalous pulse identification, anomalous pulse filtering, anomalous pulse removal, beat segmentation, or any combination thereof, such as described in greater detail elsewhere herein. According to some embodiments, the method may include analyzing at least one signal and/or segment using frequency domain and/or pulse domain, such as described in greater detail elsewhere herein.

According to some embodiments, the method 1200 may include inputting the first signal to one or more algorithms configured to predict the estimated arterial pressure waveform. According to some embodiments, the method 1200 may include inputting the first signal, the preprocessed signal, and/or segments associated with the first signal, to the one or more algorithms. According to some embodiments, the input to the one or more algorithms may be in the form of raw signals, preprocessed signals, and/or as one or more features thereof.

According to some embodiments, at step 1204, the method may include building and/or configuring a patient model based at least in part, on the first arterial pressure waveform of the patient.

According to some embodiments, building and/or configuring the patient model may include one or more algorithms configured to receive the inputted first signal. According to some embodiments, the model may include a cardiovascular physiological model. According to some embodiments, the model may include any one or more of a Windkessel model, 0D models, 1D models, 2D models, one or more simulations of blood system, hemodynamic models, averaged expected response calculations, mathematical models, or any combination thereof. According to some embodiments, the model may be devoid of machine learning (or artificial intelligent) algorithms.

According to some embodiments, building and/or configuring the patient model may include a machine learning algorithm (or in other words, machine learning model). According to some embodiments, the machine learning algorithm may include any one or more of one or more regression models, neural networks, (deep) convolutional networks, support vector regressor, or any combination thereof. According to some embodiments, the machine learning model may be trained to estimate the effects of medication (of a CMAP) after medication administration. According to some embodiments, the machine learning model may be trained to estimate the effects of medication (of a CMAP) and output the estimated arterial waveform.

According to some embodiments, the method may include receiving metadata associated with demographic data and/or medical history of the patient. According to some embodiments, the metadata associated with demographic data and/or medical history of the patient may be inputted by the user (i.e., the patient and/or the physician). According to some embodiments, the metadata associated with demographic data and/or medical history of the patient may be retrieved automatically from healthcare database (EHR-Electronic health record). According to some embodiments, the model may be configured to receive and/or store metadata associated with demographic data and/or medical history of the patient. According to some embodiments, the metadata may include any one or more of the age, gender, chronic medical conditions, current medical conditions, currently administered medications (and/or dosage thereof), and the like.

According to some embodiments, at step 1206, the method may include predicting, using the model of step 1204, and a prediction algorithm an estimated arterial pressure waveform in response to the change in cardiovascular medication administration program. According to some embodiments, the output of the estimated arterial pressure waveform may be in the form of raw signals, preprocessed-like signals, and/or as one or more features thereof. According to some embodiments, the prediction may be configured to generate the estimated arterial pressure waveform in response to the change in cardiovascular medication administration program based, at least in part, on the first arterial pressure waveform of the patient (or the first signal) and the type of change in cardiovascular medication administration program.

According to some embodiments, the prediction algorithm may include one or more of the following: adjusting the physiological model parameters based on the proposed CMAP, a machine learning prediction algorithm (or in other words, machine learning regression model). According to some embodiments, the machine learning algorithm may include any one or more of one or more regression models, neural networks, (deep) convolutional networks, support vector regressor, or any combination thereof. According to some embodiments, the machine learning model may be trained to estimate the effects of medication (of a CMAP) on the subject's physiological model after medication administration and adjusting the physiological model parameters. According to some embodiments, the machine learning model may be trained to estimate the effects of medication (of a CMAP) after medication administration. According to some embodiments, the machine learning model may be trained to estimate the effects of medication (of a CMAP) and output the estimated arterial waveform.

According to some embodiments, the prediction algorithm may be configured to predict the estimated arterial pressure waveform in response to the change in cardiovascular medication administration program (or in other words, the intended change in CMAP) using the metadata associated with demographic data and/or medical history of the patient.

According to some embodiments, the method may include receiving data associated with the intended medication and/or dosage thereof. According to some embodiments, the model may be configured to predict the estimated arterial pressure waveform in response to an intended change in cardiovascular medication administration program. According to some embodiments, the model may be configured to predict the estimated arterial pressure waveform in response to an intended change in CMAP based, at least in part, on the data associated with the intended medication and/or dosage thereof.

According to some embodiments, at step 1208, the method may include receiving at least one second signal associated with a second arterial pressure waveform of the patient obtained after the initiation of change in cardiovascular medication administration program. According to some embodiments, receiving the at least one second signal may include recording the at least one second signal. According to some embodiments, recording may refer to a single blood pressure waveform acquisition of the second signal. According to some embodiments, recording may refer to a blood pressure waveform acquisition over a short period of time (such as, for example, several seconds and/or several heartbeats). According to some embodiments, the recording may be blood pressure waveform acquisition for one or more seconds. According to some embodiments, recording may refer to a (continuous) blood pressure waveform acquisition over long time (such as, for example, a range from one or more minutes to a plurality of days). According to some embodiments, recording may refer to multiple recordings, which may include one or more recordings over short periods of time, one or more recordings over long periods of time and/or any combination thereof.

According to some embodiments, the time difference between initiation of the change in cardiovascular medication administration program and the time in which the at least one second signal is obtained is lower than 30 seconds. According to some embodiments, the time difference between initiation of the change in cardiovascular medication administration program and the time in which the at least one second signal is obtained is lower than 60 seconds.

According to some embodiments, the time difference between initiation of the change in cardiovascular medication administration program and the time in which the at least one second signal is obtained is arbitrary. According to some embodiments, the time difference between initiation of the change in cardiovascular medication administration program and the time in which the at least one second signal is obtained is dependent on the type of medication. According to some embodiments, the time difference between initiation of the change in cardiovascular medication administration program and the time in which the at least one second signal is obtained is arbitrary within a short range, of about 30 seconds to 3 minutes. According to some embodiments, the time difference between initiation of the change in cardiovascular medication administration program and the time in which the at least one second signal is obtained is arbitrary within a long range, of about 1 to 7 days. According to some embodiments, the time difference between initiation of the change in cardiovascular medication administration program and the time in which the at least one second signal is obtained varies and correlates to a type of medication associated with the cardiovascular medication administration program.

According to some embodiments, the method 1200 may include preprocessing the second signal. According to some embodiments, the preprocessing may include any one or more of noise detection, noise reduction, noise cancellation, signal enhancement, motion compensation, blood pressure calculation, signal quality assessment, pulse detection, pulse normalization, heartbeat duration calculation, time normalization, anomalous pulse detection, anomalous pulse identification, anomalous pulse filtering, anomalous pulse removal, beat segmentation, or any combination thereof, such as described in greater detail elsewhere herein. According to some embodiments, method may include analyzing at least one signal and/or segment using frequency domain and/or pulse domain, such as described in greater detail elsewhere herein.

According to some embodiments, at step 1210, the method may include comparing the predicted estimated arterial pressure waveform and the at least one second signal, thereby identifying differences therebetween. According to some embodiments, comparing the predicted estimated arterial pressure waveform and the at least one second signal (and/or one or more of the plurality of segments) may include identifying one or more differences and/or classifying the one or more differences using one or more algorithms. According to some embodiments, the algorithm may be configured to compare between the predicted estimated arterial pressure waveform and the at least one second signals (and/or one or more of the plurality of segments). According to some embodiments, the algorithm may be configured to classify the differences as being associated with an effective result of the CMAP. According to some embodiments, the algorithm may be configured to classify the differences as being associated with a positive effective result of the CMAP, a negative effective result of the CMAP, a placebo effect result of the CMAP, and no effective result of the CMAP. According to some embodiments, the algorithm may include one or more machine learning algorithms. According to some embodiments, the algorithm may be configured to score (or rank) the quality of change between the predicted estimated arterial pressure waveform and the at least one second signals (and/or one or more of the plurality of segments). According to some embodiments, the algorithm may be configured to assess the one or more differences between the predicted estimated arterial pressure waveform and the at least one second signal (and/or one or more of the plurality of segments).

It is understood that the term “one or more algorithms” may include a single algorithm or a plurality of algorithms. According to some embodiments, one algorithm may include therein a plurality of algorithms.

According to some embodiments, at step 1212, the method may include displaying the identified differences between the predicted estimated arterial pressure waveform and the at least one second signal.

According to some embodiments, the model may be configured to output a recommended cardiovascular medication administration. According to some embodiments, the method may include outputting a recommended cardiovascular medication administration program (or in other words, a suggestion) based, at least in part, on the estimated arterial pressure waveform and/or the at least one second signal. According to some embodiments, the method may include outputting a suggestion for a change in CMAP based, at least in part, on the comparison of the estimated arterial pressure waveform and the at least one second signal. According to some embodiments, the type of change in cardiovascular medication administration program includes any one or more of: beginning a new treatment, change in type of medication, change in dosage of a medication, and change in timing of a medication, changing an administration regime, or any combination thereof.

According to some embodiments, the model may be configured to detect a placebo effect of the CMAP on the patient. According to some embodiments, the model may be configured to identify one or more features of the second signal which may be associated with the placebo effect. For example, if the blood pressure of the patient is lower in the second signal than in the first signal, but the medication is not effective. According to some embodiments, the model may be configured to detect if the medication is not effective. According to some embodiments, the model may be configured to detect if the medication effects the patient negatively (or in other words, the CMAP resulted in an undesired result or damage). For example, if the patient had received blood pressure medication (that should lower their blood pressure by dilating the arteries) that lowered the patient's blood pressure, and the second signal shows one or more differences in the signal heart beats' shape, the model may detect that the differences are associated with dilated arteries suggesting that the medication is effective. Another example, if the patient had received blood pressure medication (that should lower their blood pressure by dilating the arteries) that lowered the patient's blood pressure, and the second signal shows one or more differences in the signal heart beats' shape, the model may detect that the differences may not necessarily be associated with lower blood pressure thereby indicating a placebo effect.

According to some embodiments, the method includes displaying the differences using one or more visual representation techniques. According to some embodiments, the one or more visual representation techniques may be selected from: a two-dimensional attractor, graphs of waveform signals, graphs of the difference in the signals, attractor reconstruction, Hilbert-Huang spectrum analysis with empirical mode decomposition (EMD), and the difference between waveform characteristics. For example, according to some embodiments, the visual representation techniques may include displaying the normalized pulse waveform after taking the medication and the predicted estimated waveform.

According to some embodiments, the method may include displaying one or more of the received waveform signals and/or the predicted estimated waveform together (such as, for example, on the same graph). According to some embodiments, the method may include displaying the received waveform signals and/or the predicted estimated waveform separately (such as, for example, on separate, individual graphs) and display the difference in the signals (e.g., subtracted signal).

According to some embodiments, displaying the identified differences may include using any one or more of pulse analysis methods, such as, for example, attractor reconstruction, or Hilbert-Huang spectrum analysis with empirical mode decomposition (EMD), or any combination thereof. According to some embodiments, displaying the identified differences between the received waveform signals (i.e., at least one of the first and the second signals) and/or the predicted estimated waveform may include displaying the difference between waveform characteristics (such as, for example, using graphs or bars).

According to some embodiments, displaying the identified differences between the received waveform signals and/or the predicted estimated waveform may include displaying changes to one or more individual features, such as, for example, a change in variability between pulses over time of the first and/or second signals (or the plurality of segments thereof).

According to some embodiments, the displaying may include a two-dimensional (2D) representation for identifying changes in the shape (or in other words, an attractor reconstruction). According to some embodiments, the 3D attractor may be viewed as a 2D attractor, as described in greater details elsewhere herein.

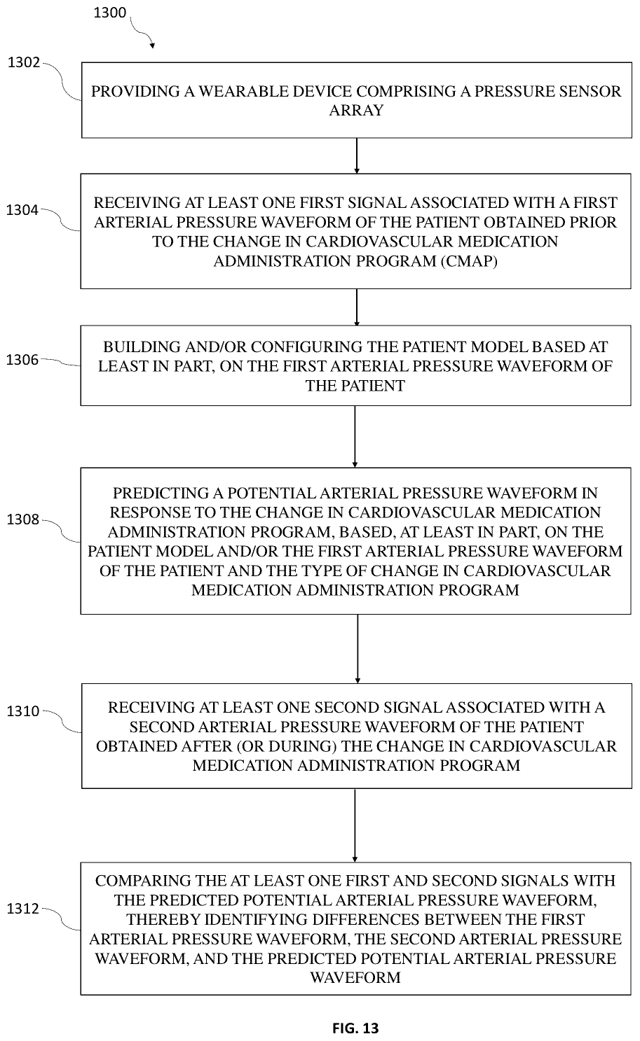

Reference is made to FIG. 13, which shows a flow chart of the steps of a method for predicting and evaluating the response to and/or the effectiveness of a cardiovascular medication administration program using a physiological model, in accordance with some embodiments of the present invention. According to some embodiments, the methods 300/1100/1200 may include one or more steps of the method 1300. According to some embodiments, the method 1300 may include one or more steps of methods 300/1100/1200.

According to some embodiments, at step 1302, the method 1300 may include providing a wearable device including a pressure sensor array. According to some embodiments, at step 1304, the method 1300 may include receiving at least one first signal associated with a first arterial pressure waveform of the patient obtained prior to the change in cardiovascular medication administration program (CMAP). According to some embodiments, at step 1306, the method 1300 may include building and/or configuring the patient model based at least in part, on the first arterial pressure waveform of the patient. According to some embodiments, at step 1308, the method 1300 may include predicting an estimated arterial pressure waveform in response to the change in cardiovascular medication administration program, based, at least in part, on the patient model and/or the first arterial pressure waveform of the patient and the type of change in cardiovascular medication administration program. According to some embodiments, at step 1310, the method may include receiving at least one second signal associated with a second arterial pressure waveform of the patient obtained after (or during) the change in cardiovascular medication administration program. According to some embodiments, at step 1312, the method may include comparing the at least one first and second signals with the predicted estimated arterial pressure waveform, thereby identifying differences between the first arterial pressure waveform, the second arterial pressure waveform, and the predicted estimated arterial pressure waveform.

According to some embodiments, at step 1302, the method 1300 may include providing a wearable device including a pressure sensor array (such as, for example the device 100 of FIG. 1A and FIG. 1B). According to some embodiments, the method may include receiving at least one first signal and/or second signal from a wearable device, such as, device 100. According to some embodiments, the processor of device 100 may be configured to execute method 1300.

According to some embodiments, at step 1304, the method 1300 may include receiving at least one first signal associated with a first arterial pressure waveform of the patient obtained prior to the change in cardiovascular medication administration program (CMAP).

According to some embodiments, receiving the at least one first signal may include recording the at least one first signal. According to some embodiments, recording may refer to a single blood pressure waveform acquisition of the first signal. According to some embodiments, recording may refer to a blood pressure waveform acquisition over a short period of time (such as, for example, several seconds and/or several heartbeats).

According to some embodiments, the recording may be blood pressure waveform acquisition for one or more seconds. According to some embodiments, recording may refer to a (continuous) blood pressure waveform acquisition over long time (such as, for example, a range from one or more minutes to a plurality of days). According to some embodiments, recording may refer to multiple recordings, which may include one or more recordings over short periods of time, one or more recordings over long periods of time and/or any combination thereof.

According to some embodiments, the method 1300 may include preprocessing the first signal. According to some embodiments, the preprocessing may include any one or more of noise detection, noise reduction, noise cancellation, signal enhancement, motion compensation, blood pressure calculation, signal quality assessment, pulse detection, pulse normalization, heartbeat duration calculation, time normalization, anomalous pulse detection, anomalous pulse identification, anomalous pulse filtering, anomalous pulse removal, beat segmentation, or any combination thereof, such as described in greater detail elsewhere herein. According to some embodiments, the method 1300 may include analyzing at least one signal and/or segment using frequency domain and/or pulse domain, such as described in greater detail elsewhere herein.

According to some embodiments, the method 1300 may include inputting the first signal to one or more algorithms configured to predict the estimated arterial pressure waveform. According to some embodiments, the method 1300 may include inputting the first signal, the preprocessed signal, and/or segments associated with the first signal, to the one or more algorithms. According to some embodiments, the input to the one or more algorithms may be in the form of raw signals, preprocessed signals, and/or as one or more features thereof.

According to some embodiments, at step 1306, the method may include building and/or configuring the patient model based at least in part, on the first arterial pressure waveform of the patient. According to some embodiments, building and/or configuring the patient model may include one or more algorithms configured to receive the inputted first signal. According to some embodiments, the model may include a cardiovascular physiological model. According to some embodiments, the model may include any one or more of a Windkessel model, 0D models, 1D models, 2D models, one or more simulations of blood system, hemodynamic models, averaged expected response calculations, mathematical models, or any combination thereof. According to some embodiments, the model may be devoid of machine learning (or artificial intelligent) algorithms.

According to some embodiments, building and/or configuring the patient model may include a machine learning algorithm (or in other words, machine learning model). According to some embodiments, the machine learning algorithm may include any one or more of one or more regression models, neural networks, (deep) convolutional networks, support vector regressor, or any combination thereof. According to some embodiments, the machine learning model may be trained to estimate the effects of medication (of a CMAP) after medication administration. According to some embodiments, the machine learning model may be trained to estimate the effects of medication (of a CMAP) and output the estimated arterial waveform.

According to some embodiments, the method may include receiving metadata associated with demographic data and/or medical history of the patient. According to some embodiments, the metadata associated with demographic data and/or medical history of the patient may be inputted by the user (i.e., the patient and/or the physician). According to some embodiments, the metadata associated with demographic data and/or medical history of the patient may be retrieved automatically from healthcare database (EHR-Electronic health record). According to some embodiments, the model may be configured to receive and/or store metadata associated with demographic data and/or medical history of the patient. According to some embodiments, the metadata may include any one or more of the age, gender, chronic medical conditions, current medical conditions, currently administered medications (and/or dosage thereof), and the like.

According to some embodiments, at step 1308, the method may include predicting, using the model of step 1306, and a prediction algorithm an estimated arterial pressure waveform in response to the change in cardiovascular medication administration program. According to some embodiments, the output of the estimated arterial pressure waveform may be in the form of raw signals, preprocessed-like signals, and/or as one or more features thereof. According to some embodiments, the prediction may be configured to generate the estimated arterial pressure waveform in response to the change in cardiovascular medication administration program based, at least in part, on the first arterial pressure waveform of the patient (or the first signal) and the type of change in cardiovascular medication administration program.

According to some embodiments, the prediction algorithm may include one or more of the following: adjusting the physiological model parameters based on the proposed CMAP, a machine learning prediction algorithm (or in other words, machine learning regression model). According to some embodiments, the machine learning algorithm may include any one or more of one or more regression models, neural networks, (deep) convolutional networks, support vector regressor, or any combination thereof. According to some embodiments, the machine learning model may be trained to estimate the effects of medication (of a CMAP) on the subject's physiological model after medication administration and adjusting the physiological model parameters. According to some embodiments, the machine learning model may be trained to estimate the effects of medication (of a CMAP) after medication administration. According to some embodiments, the machine learning model may be trained to estimate the effects of medication (of a CMAP) and output the estimated arterial waveform.

According to some embodiments, the prediction algorithm may be configured to predict the estimated arterial pressure waveform in response to the change in cardiovascular medication administration program (or in other words, the intended change in CMAP) using the metadata associated with demographic data and/or medical history of the patient.

According to some embodiments, the method 1300 may include receiving data associated with the intended medication and/or dosage thereof. According to some embodiments, the model may be configured to predict the estimated arterial pressure waveform in response to an intended change in cardiovascular medication administration program. According to some embodiments, the model may be configured to predict the estimated arterial pressure waveform in response to an intended change in CMAP based, at least in part, on the data associated with the intended medication and/or dosage thereof. According to some embodiments, at step 1310, the method may include receiving at least one second signal associated with a second arterial pressure waveform of the patient obtained after (or during) the change in cardiovascular medication administration program.

According to some embodiments, receiving the at least one second signal may include recording the at least one second signal. According to some embodiments, recording may refer to a single blood pressure waveform acquisition of the second signal. According to some embodiments, recording may refer to a blood pressure waveform acquisition over a short period of time (such as, for example, several seconds and/or several heartbeats). According to some embodiments, the recording may be blood pressure waveform acquisition for one or more seconds. According to some embodiments, recording may refer to a (continuous) blood pressure waveform acquisition over long time (such as, for example, a range from one or more minutes to a plurality of days). According to some embodiments, recording may refer to multiple recordings, which may include one or more recordings over short periods of time, one or more recordings over long periods of time and/or any combination thereof.

According to some embodiments, the time difference between initiation of the change in cardiovascular medication administration program and the time in which the at least one second signal is obtained is lower than 30 seconds. According to some embodiments, the time difference between initiation of the change in cardiovascular medication administration program and the time in which the at least one second signal is obtained is lower than 60 seconds.

According to some embodiments, the time difference between initiation of the change in cardiovascular medication administration program and the time in which the at least one second signal is obtained is arbitrary. According to some embodiments, the time difference between initiation of the change in cardiovascular medication administration program and the time in which the at least one second signal is obtained is dependent on the type of medication. According to some embodiments, the time difference between initiation of the change in cardiovascular medication administration program and the time in which the at least one second signal is obtained is arbitrary within a short range, of about 30 seconds to 3 minutes. According to some embodiments, the time difference between initiation of the change in cardiovascular medication administration program and the time in which the at least one second signal is obtained is arbitrary within a long range, of about 1 to 5 days. According to some embodiments, the time difference between initiation of the change in cardiovascular medication administration program and the time in which the at least one second signal is obtained varies and correlates to a type of medication associated with the cardiovascular medication administration program.

According to some embodiments, the method 1300 may include preprocessing the second signal. According to some embodiments, the preprocessing may include any one or more of noise detection, noise reduction, noise cancellation, signal enhancement, motion compensation, blood pressure calculation, signal quality assessment, pulse detection, pulse normalization, heartbeat duration calculation, time normalization, anomalous pulse detection, anomalous pulse identification, anomalous pulse filtering, anomalous pulse removal, beat segmentation, or any combination thereof, such as described in greater detail elsewhere herein. According to some embodiments, method may include analyzing at least one signal and/or segment using frequency domain and/or pulse domain, such as described in greater detail elsewhere herein.

According to some embodiments, at step 1312, the method may include comparing the at least one first and/or second signals with the predicted estimated arterial pressure waveform, thereby identifying differences between the first arterial pressure waveform and/or the second arterial pressure waveform, and the predicted estimated arterial pressure waveform.

According to some embodiments, comparing the at least one first and/or second signals with the predicted estimated arterial pressure waveform may include identifying one or more differences and/or classifying the one or more differences using one or more algorithms. According to some embodiments, the algorithm may be configured to compare between the at least one first and/or second signals with the predicted estimated arterial pressure waveform. According to some embodiments, the algorithm may be configured to classify the differences as being associated with an effective result of the CMAP. According to some embodiments, the algorithm may be configured to classify the differences as being associated with a positive effective result of the CMAP, a negative effective result of the CMAP, a placebo effect result of the CMAP, and no effective result of the CMAP. According to some embodiments, the algorithm may include one or more machine learning algorithms. According to some embodiments, the algorithm may be configured to score (or rank) the quality of change between the at least one first and/or second signals with the predicted estimated arterial pressure waveform. According to some embodiments, the algorithm may be configured to assess the one or more differences between the at least one first and/or second signals with the predicted estimated arterial pressure waveform.

According to some embodiments, the method may include displaying the identified differences between the first arterial pressure waveform, the second arterial pressure waveform, and the predicted estimated arterial pressure waveform. According to some embodiments, the method includes displaying using one or more visual representation techniques, such as described in greater detail elsewhere herein.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to case understanding of the specification and should not be construed as necessarily limiting.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an crasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer (or cloud) may be connected to the user's computer through any type of network, including a local area network (LAN) or a wide area network (WAN), or the connection may be made to an external computer (for example, through the Internet using an Internet Service Provider) including wired or wireless connection (such as, for example, Wi-Fi, BT, mobile, and the like). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1.-40. (canceled)

41. A method for evaluating the response to and/or the effectiveness of a cardiovascular medication administration program, the method comprising:
receiving at least one first signal associated with a first arterial pressure waveform of the patient obtained prior to the change in cardiovascular medication administration program;
predicting, using a model, an estimated arterial pressure waveform in response to the change in cardiovascular medication administration program, based, at least in part, on the first arterial pressure waveform of the patient and the type of change in cardiovascular medication administration program;
receiving at least one second signal associated with a second arterial pressure waveform of the patient obtained after the initiation of change in cardiovascular medication administration program;
comparing the predicted estimated arterial pressure waveform and the at least one second signal, thereby identifying differences therebetween; and
displaying the identified differences.

42. The method of claim 41, further comprising outputting a suggestion for a change in CMAP based, at least in part, on the comparison of the estimated arterial pressure waveform and the at least one second signal.

43. The method of claim 41, wherein the at least one first signal and/or the at least one second signal is continuous.

44. The method claim 41, wherein the type of change in cardiovascular medication administration program comprises any one or more of: beginning a new treatment, change in type of medication, change in dosage of a medication, and change in timing of a medication, changing an administration regime, or any combination thereof.

45. The method of claim 41, further comprising preprocessing the at least one first signal and/or the at least one second signal and/or the predicted estimated arterial pressure waveform, wherein the preprocessing comprises: dividing at least a portion of the least one signal into a plurality of segments, wherein each segment comprises a heart cycle and/or calculating the blood pressure from the at least one first signal and/or the at least one second signal and/or the predicted estimated arterial pressure waveform.

46. The method of claim 41, further comprising normalizing the at least one signal and/or the at least one second signal and/or the predicted estimated arterial pressure waveform based, at least in part, on the blood pressure.

47. The method of claim 41, further comprising normalizing the at least one signal and/or the at least one second signal and/or the predicted estimated arterial pressure waveform based, at least in part, on a length of time between beats.

48. The method of claim 41, wherein estimating the estimated arterial pressure waveform is based, at least in part, on: metadata associated with demographic data and/or medical history of the patient; and/or on the intended medication and/or dosage of the cardiovascular medication administration program.

49. The method of claim 41, wherein the model comprises a cardiovascular physiological model.

50. The method of claim 50, wherein the cardiovascular physiological model comprises any one or more of a Windkessel model, 0D models, 1D models, 2D models, one or more simulations of blood system, hemodynamic models, averaged expected response calculations, mathematical models, or any combination thereof.

51. The method of claim 41, wherein the model comprises a machine learning model.

52. The method of claim 41, further comprising displaying the first signal and/or the predicted arterial pressure waveform using one or more visual representation techniques.

53. The method claim 52, wherein the visual representation techniques are selected from: a two dimensional attractor, graphs of waveform signals, graphs of the difference in the signals, attractor reconstruction, Hilbert-Huang spectrum analysis with empirical mode decomposition (EMD), and the difference between waveform characteristics.

54. The method of claim 41, wherein the time difference between initiation of the change in cardiovascular medication administration program and the time in which the at least one second signal is obtained is lower than 30 seconds.

55. The method of claim 41, wherein the time difference between initiation of the change in cardiovascular medication administration program and the time in which the at least one second signal is obtained is arbitrary.

56. The method of claim 41, wherein the time difference between initiation of the change in cardiovascular medication administration program and the time in which the at least one second signal is obtained varies and correlates to a type of medication associated with the cardiovascular medication administration program.

57. The method of claim 41, wherein the medication associated with the cardiovascular medication administration program comprises any one or more of one or more artery dilators (Vasodilators), beta blockers, Alpha blockers, Calcium channel blockers, Diuretics, Angiotensin-converting enzyme (ACE) inhibitors, Angiotensin receptor blockers (ARBs), Central Agonists, or any combination thereof.

58. A device for evaluating the response to and/or the effectiveness of a cardiovascular medication administration program, the device comprising:
a wearable body comprising a pressure sensor array and configured to be worn by a patient; and
a processor in communication with a non-transitory computer-readable storage medium, the storage medium has stored thereon one or more program codes executable by the processor to:
receive at least one first signal associated with a first arterial pressure waveform of the patient obtained prior to the change in cardiovascular medication administration program;
predict, using a model, an estimated arterial pressure waveform in response to the change in cardiovascular medication administration program, based, at least in part, on the first arterial pressure waveform of the patient and the type of change in cardiovascular medication administration program;
receive at least one second signal associated with a second arterial pressure waveform of the patient obtained after the initiation of change in cardiovascular medication administration program; and
compare the predicted estimated arterial pressure waveform and the at least one second signal, thereby identifying differences therebetween.

59. A method for predicting and evaluating the response to and/or the effectiveness of a cardiovascular medication administration program (CMAP), the method comprising:
providing a wearable device comprising a pressure sensor array;
receiving at least one first signal associated with a first arterial pressure waveform of the patient obtained prior to the change in CMAP;
predicting, using a model, an estimated arterial pressure waveform in response to the change in cardiovascular medication administration program, based, at least in part, on the first arterial pressure waveform of the patient and the type of change in CMAP;
receiving at least one second signal associated with a second arterial pressure waveform of the patient obtained after (or during) the change in CMAP;
comparing the at least one first and second signals with the predicted estimated arterial pressure waveform, thereby identifying differences between the first arterial pressure waveform, the second arterial pressure waveform, and the predicted estimated arterial pressure waveform; and
displaying the identified differences between the first arterial pressure waveform, the second arterial pressure waveform, and the predicted estimated arterial pressure waveform.

60. A device for predicting and evaluating the response to and/or the effectiveness of a cardiovascular medication administration program (CMAP), the device comprising:
a wearable body comprising a pressure sensor array and configured to be worn by a patient; and
a processor in communication with a non-transitory computer-readable storage medium, the storage medium has stored thereon one or more program codes executable by the processor to execute the method of claim 59.
